(19) United States
(12) Patent Application Publication
(10) Pub. No.: US 2011/0245283 A1
(43) Pub. Date: Oct. 6, 2011

(54) Methods for predicting the response to statins

(76) Inventor: Glen H. Murata, Albuquerque, NM (US)

(21) Appl. No.: 12/928,894

(22) Filed: Dec. 21, 2010

Related U.S. Application Data
(60) Provisional application No. 61/284,494, filed on Dec. 21, 2009.
(60) Provisional application No. 61/284,497, filed on Dec. 21, 2009.

(51) Int. Cl. A61K 31/505; A61K 31/40; A61K 31/404; A61K 31/366; A61K 31/22; A61K 31/47; A61P 9/10; G06G 7/60

(52) U.S. Cl. 514/275; 514/423; 514/419; 514/460; 514/510; 514/311; 703/11

(57) ABSTRACT

The invention provides methods for optimizing therapeutic efficacy for treating hypercholesterolemia in a subject having a cardiovascular disease (CVD), comprising (a) determining subject characteristics that affect the likelihood of reaching a goal level of low density lipoprotein (LDL); and (b) obtaining success probabilities of a variety of statin treatments for reaching said goal level of LDL using said subject characteristics and a multivariate model; and (c) administrating the optimal statin treatment with the highest success probability of step (b) to said subject thereby optimizing therapeutic efficacy for treating hypercholesterolemia in said subject.

This patent application claims the benefit of the filing dates of U.S. Ser. No. 61/284,497, filed Dec. 21, 2009, and U.S. Ser. No. 61/284,494, filed Dec. 21, 2009. The contents of all of the foregoing applications are incorporated by reference in their entireties into the present patent application.

Throughout this application, various publications are referenced. The disclosures of these publications are hereby incorporated by reference herein in their entireties.

### FIELD OF THE INVENTION

This invention relates generally to statins and, specifically, to predicting what statin to use in treatment.

### BACKGROUND OF THE INVENTION

The statins are the most commercially successful class of drugs in the history of the pharmaceutical industry. They are remarkably effective when used for the primary and secondary prevention of cardiovascular disease (CVD). They are indicated for patients with coronary disease, stroke, and diabetes (3 of the 6 most common causes of death among US residents) and for reducing the risk of CVD in those with hypertension and tobacco use (which affect 29% and 21%, respectively, of US adults). Their side-effects are so well tolerated that simvastatin has been available without prescription in Great Britain since 2004. These factors explain why atorvastatin generated $13.6 billion in sales in 2006—making it the best selling drug ever.

Despite these characteristics, up to half of CVD patients on statins do not reach their goals for low density lipoprotein (LDL). These dismal results have been attributed to low adherence by patients and to “clinical inertia” by providers. Extensive studies of the latter phenomenon have been done and found that poor performance is actually due to limited opportunities for treatment and poor statin selection. In one study of 2,699 CVD patients making 3,768 visits to their primary care providers, no action was taken in 67.1% of visits where the LDL was above the recommended target. Moreover, for patients not at goal, providers managed to make only 2.10±1.14 changes in treatment over 2.81±2.37 years of follow-up. Hierarchical regression identified multiple barriers at the visit, patient, provider, and institutional levels. In a second study, it was found that no dose adjustment involving lovastatin was more than 50% successful, while no change involving simvastatin was more than 62% successful. These observations suggest that there is a critical need for an objective method of choosing treatment that will result in goal within 1 or 2 attempts. Moreover, these models should be derived from real world experiences to maximize their relevance to clinical practice and be rigorously validated. This application is intended to protect the results of studies that have achieved these goals.

### SUMMARY OF THE INVENTION

The invention provides method for optimizing the therapeutic efficacy for treating hypercholesterolemia in a subject having a cardiovascular disease (CVD). In one embodiment, the method comprises the steps of determining subject characteristics that affect the likelihood of reaching a goal level of low density lipoprotein (LDL); and obtaining success probabilities of a variety of statin treatments for reaching said goal level of LDL using said subject characteristics and a multivariate model. After an optimal statin treatment having the highest success probability is identified and/or chosen then the method comprises administrating the optimal statin treatment to the subject thereby optimizing the therapeutic efficacy for treating hypercholesterolemia in the subject.

Additionally, the invention provides methods for predicting the success probability of a statin treatment in a subject having a CVD. This method comprises determining levels of characteristics of said subject; and applying the levels of characteristics through a multivariate model to obtain the success probability of said statin treatment in said subject.

### BRIEF DESCRIPTION OF THE DRAWINGS

FIG. 1 shows the clinical pathway of the dose titration module.

FIG. 2 shows the drug-maintenance module.

FIG. 3 shows the titration rules for Fluvastatin, Lovastatin, and Atorvastatin.

FIG. 4 shows the process used to assure that subjects have logged on at the appropriate times.

### DETAILED DESCRIPTION OF THE INVENTION DEFINITION

As used in this application, the following words or phrases have the meanings specified.

As used herein, “hypercholesterolemia” refers to the presence of excess cholesterol in the blood.

It is noted that, as used in this specification and the appended claims, the singular forms “a,” “an,” and “the,” include plural referents unless expressly and unequivocally limited to one referent. Thus, for example, reference to “a statin” includes two or more different statins. As used herein, the term “include” and its grammatical variants are intended to be non-limiting, such that recitation of items in a list is not to the exclusion of other like items that can be substituted or other items that can be added to the listed items.

In order that the invention herein described may be more fully understood, the following description is set forth.

The invention provides method for optimizing the therapeutic efficacy for treating hypercholesterolemia in a subject having a cardiovascular disease (CVD). In an embodiment of the invention, the method comprises the steps of determining subject characteristics that affect the likelihood of reaching a goal level of low density lipoprotein (LDL); and inputting the subject characteristics into a multivariate model to generate success probabilities of a variety of statin treatments for reaching said goal level of LDL. After an optimal statin treatment having the highest success probability is identified and/or chosen then the method comprises administrating the optimal statin treatment to the subject thereby optimizing the therapeutic efficacy for treating hypercholesterolemia in the subject. In one embodiment of the invention, the subject is on a statin regimen. In another embodiment, the subject is not on a statin regimen.

This invention provides a method of optimizing therapeutic efficacy of treatment for lowering the level of LDL in a subject. The method comprises the following steps: first, subject characteristics that affect the likelihood of reaching a goal level of LDL are determined; second, said subject characteristics are inputted into a multivariate model to generate success probabilities of a variety of statin treatments for reaching said goal level of LDL; finally, among said statin treatments, the optimal statin treatment with the highest success probability is administrated to said subject thereby optimizing therapeutic efficacy for treating hypercholesterolemia in said subject.

Additionally, the invention provides methods for predicting the success probability of a statin treatment in a subject having a CVD. This method comprises determining the subject characteristics that affect the likelihood of reaching a goal level of LDL; and applying or inputting the subject characteristics into a multivariate model to obtain success probabilities of a variety of statin treatments for reaching said goal level of LDL in said subject.

In accordance with the practice of the invention, CVD is a disease involving the heart or blood vessels including but not limited to atherosclerosis, coronary artery disease (CAD), diabetes, cerebrovascular disease, aortic or large vessel disease, peripheral vascular disease, angina, arrhythmia, and cardiomyopathy.

As used herein, a statin is a 3-hydroxy-3-methylglutaryl-coenzyme A (HMG-CoA) reductase inhibitor and belongs to a class of drugs generally used to lower cholesterol levels by inhibiting the enzyme HMG-CoA reductase. Suitable examples of a statin include but are not limited to atorvastatin, ceruvostatin, fluvastatin, lovastatin, osuvastatin, pravastatin, pitavastatin, rosuvastatin, simvastatin, or a combination thereof.

An optimal statin treatment is a treatment that involves the use of a statin that provides a desired goal level of LDL. The goal level may be determined by a physician or a patient. In accordance with the practice of the invention, to reach a goal level of LDL, many statin treatments are possible. For example, in order to choose the treatment with the highest possibility of success, the multivariate model may be used to calculate the success probabilities (e.g., predicted probability of attaining goal) of various possible statin treatments, and the one with the highest success probability maybe determined as the optimal statin treatment.

For example, statin treatments for a subject previously or currently treated with a statin include treatments applying the same statin at a different (e.g., higher) dose, a different type of statin with the same dose, a different type of statin with a different dose, or a combination thereof.

In accordance with the practice of the invention, statins may be administered in dosages that include but are not limited to 10 mg, 20 mg, 30 mg, 40 mg, 50 mg, 60 mg, 70 mg, 80 mg. The most effective mode of administration and dosage regimen for the statins of the present invention depends upon the severity and course of the disease, the subject's health and response to treatment and the judgment of the treating physician. Accordingly, the dosages of the molecules should be titrated to the individual subject.

The interrelationship of dosages for animals of various sizes and species and humans based on mg/m² of surface area is described by Freireich, E. J., et al. (Quantitative Comparison of Toxicity of Anticancer Agents in Mouse, Rat, Hamster, Dog, Monkey and Man. Cancer Chemother, Rep., 50, No. 4, 219-244, May 1966).

In accordance with the practice of the invention, the multivariate model may be constructed using a process comprising steps of determining levels of characteristics of a CAD cohort; assembling a predictor file; creating a response file; linking the predictor file and the response file; and deriving said multivariate model.

Also in accordance with the practice of the invention, the subject characteristics (also referred to herein as covariates for the multivariate model (merely by way of example, see Sections III, IV, V and VI) or predictors) include, but are not limited to, one or more of the subject's age, gender, BMI, HBA1C (hemoglobin A1c), preceding LDL levels, prior statin use, prior statin dose, and/or frequency of prior statin use. Additionally, subject characteristics useful in the methods of the invention include the doses of one or more drugs that are administered or prescribed to the subject alone or together with a statin. Such drugs include antiarrhythmic agents (medication used for irregular heart beat) such as amiodarone; anticonvulsant and mood stabilizing drugs such as carbemazepine; thiazolidinedione (TZD) with hypoglycemic (antihyperglycemic, antidiabetic) action such as pioglitazone; calcium channel blockers such as diltiazem and verapamil; anti-diabetic drugs such as metformin and rosiglitazone; class 1b antiarrhythmic agents such as phenyloin; and antipsychotic drugs such as risperidone.

Further in one embodiment of the invention, the goal level of LDL is less than 125 mg/dl. In still another embodiment, the goal level of LDL is less than 100 mg/dl. In one embodiment, the goal level of LDL is less than 75 mg/dl. In one embodiment, the goal level of LDL is less than 70 mg/dl. In another embodiment of the invention, the goal level of LDL can be, for example, in the range of about 20-100 mg/dl. In a further embodiment, the goal level of LDL can be, for example, in the range of about 30-100 mg/dl. In yet a further embodiment, the goal level of LDL can be, for example, in the range of about 80-100 mg/dl.

In accordance with the invention, the subject may be a mammal. In other embodiments of the invention, the subject may be any of human, monkey, ape, dog, cat, cow, horse, sheep, rabbit, mouse, or rat.

In accordance with the invention, the administration of a statin treatment may be effected locally or systemically. Additionally, the route of administration of a statin treatment may be any of topical, enteral or parenteral. In other embodiments of the invention, the route of administration of a statin treatment may be any of rectal, intercisternal, bucal, intramuscular, intrasternal, intracutaneous, intrasynovial, intravenous, intraperitoneal, intraocular, periostal, intra-articular injection, infusion, oral, inhalation, subcutaneous, implantable pump, continuous infusion, gene therapy, intranasal, intrathecal, intracerebroventricular, transdermal, or by spray, patch or injection.

Upon studying the disclosure, it will be apparent to those skilled in the art that various modifications and variations can be made in the devices and methods of various embodiments of the invention. Other embodiments will be apparent to those skilled in the art from consideration of the specification and practice of the embodiments disclosed herein. It is intended that the specification and examples be considered as examples only. The various embodiments are not necessarily mutually exclusive, as some embodiments can be combined with one or more other embodiments to form new embodiments.

### EXAMPLES

### Example 1

### I. Methods

A. Selection criteria—Patients were eligible for this study if they had a diagnosis of coronary artery disease (CAD) or its equivalent; had at lease one LDL above goal after the qualifying diagnosis; were prescribed a new statin or an increased dose of an existing statin ≦365 days after the LDL was measured; and had a repeat LDL measurement no less than 42 days after statin therapy was modified. They were excluded if they were treated with any other lipid-lowering drug classes, anti-retroviral drugs, or cyclosporine at any time. All subjects treated between FY 1996 and the end of the first quarter of FY 2009 (Jan. 1, 2009) were considered for this study.

A patient was considered to have CAD or its equivalent if: 1) the patient was given a discharge diagnosis of diabetes (International Classification of Diseases, 9th Revision (ICD-9) codes 250.00-250.93), coronary artery disease (410.00-414.9), atherosclerotic cardiovascular disease (429.2), cerebrovascular disease (433.00-438.9), aortic or large vessel disease (440.0-441.9), or peripheral vascular disease (443.89-443.9); 2) any such problem was entered into the patient's problem list; 3) the patient underwent coronary artery bypass surgery (Current Procedural Terminology, 4th Edition (CPT-4) codes 33510-33514, 33516-33519, 33521-33523, 33530, or 33533-33536) or percutaneous transluminal coronary angioplasty with or without stent (92973, 92980-92982, 92984, or 92995); or 4) the patient was prescribed a medication from VA drug class HS501 (insulin) or HS502 (oral hypoglycemic agents). The date of onset was defined as the earliest of the discharge date, the date of entry onto the problem list, the procedure date, or the date that the diabetes medication was released to the patient.

B. LDL measurements—All LDL's subsequent to the date of onset were considered above goal if they were ≧100 mg/dl. The more rigorous standard of 70 mg/dl was not evaluated because certain risk factors (tobacco use and family history of premature coronary artery disease) could not be retrieved from electronic medical records. Accordingly, it was not possible to identify patients whose risk factors were uncontrolled. Prior to FY 2005, all LDL's were calculated values, and no results were reported for patients with triglyceride levels ≧400 mg/dl. Beginning in 1995, direct measurements of LDL were reported for patients whose LDL could not be calculated.

C. Statin treatment—All statins (fluvastatin, lovastatin, pravastatin, pitavastatin, osuvastatin, simvastatin, atorvastatin, ceruvostatin, and rosuvastatin) were available to providers during the study period—either as a formulary item or by special request. However, over time, several preparations were changed from non-formulary to approved status depending drug cost. The “preferred” non-formulary statin also changed with time. Because of processing costs, statins were almost always given as a single prescription as opposed to ≧2 simultaneous prescriptions. Pharmacy records contain information about the formulation (including tablet strength), quantity dispensed, days supply, and the date that the medication was given or mailed to the patient. Daily doses for all prescriptions are given by the following formula:

daily dose=(tablet strength×quantity dispensed)/days supply

A “new” statin prescription was defined as the first time that a dose or type of a statin was prescribed for each patient. New prescriptions were identified by: 1) removing “partial fills” (prescriptions for <30 days) from the prescription file; 2) doing a 4-level ascending sort of the remaining records by patient identifier, statin type, daily dose, and release date; and 3) grouping the sorted file by identifier, statin type, and statin dose, and extracting the first record.

D. Linking LDL's to prescriptions—LDL's were measured either on or off statin treatment. The former were identified by using the specimen collection and prescription release dates to link the 2 types of records. An LDL was linked to a prescription if the collection date fell between [release date] and [release date plus days supply]. Since prescriptions were often overlapping, it was possible for one measurement to be mapped to several prescriptions. Because LDL's are usually done to measure the response to the current (as opposed to previous) therapy, the LDL was assigned to the most recent prescription. This task was accomplished by: 1) doing a 2-level ascending sort of the linked records (by LDL identifier and prescription release date) and 2) grouping the sorted file by LDL identifier and extracting the last record. It was also quite common for providers to measure LDL several times on the same prescription. This practice gives the patient time to optimize non-pharmacologic therapy such as diet or exercise, improve medication adherence, and achieve stable LDL levels. For this reason, the last of several LDL's mapped to a single prescription was chosen. This task was accomplished by: 1) doing a 2-level ascending sort of the linked records (by prescription ID and specimen collection date); and 2) grouping the sorted file by prescription ID and extracting the last record. This process assured that each LDL measured on treatment was linked to the most appropriate prescription and that each prescription was linked to the LDL most likely to reflect maximal response to therapy. LDL's that could not be mapped to prescriptions were considered “off treatment”.

E. Eliminating premature LDL measurements on treatment—Although time to peak effect varies for the different statins, all achieve maximal response within 6 weeks. Therefore, linked records were eliminated if the LDL was measured prematurely. This task was accomplished by calculating the number of days between the specimen collection and prescription release dates and removing those with an interval of <42 days. It was often necessary to link the prescription in question to the immediately preceding ones to make sure that this requirement was met.

F. Linking LDL measurements above goal with new statin prescriptions—LDL measurements on and off treatment were then pooled. New statin prescriptions were then linked to their most recent LDL by: 1) calculating the interval between the specimen collection and prescription release dates; 2) doing a 2-level ascending sort by prescription ID and this interval; 3) grouping the records by prescription ID and extracting the first record; 4) removing LDL values <100 mg/dl; and 5) removing those records where the smallest interval was ≧365 days. This liberal criterion was chosen because many stable veterans are seen only once per year. This process created a record linking each new statin prescription to an immediately preceding LDL above goal and the previous dose and type of statin, if any. If also removed new prescriptions resulting from formulary changes.

G. Covariates for the multivariate model—The following list includes the predictors one or more of which may be tested in the multivariate models.
1. AGE: (expressed in units of 10 years) at time that the new statin prescription was released
2. GENDER, E. G., MALE: 1=yes, 0=no
3. BMI (kg/M²): last value before release date
4. HBA1C (hemoglobin Alc): last value before release date
5. PLDL: preceding LDL level expressed in e.g. 10 mg/dl units
6. Statins
   a. PLOVA: prior daily dose of lovastatin expressed in e.g. 10 mg units
   b. PSIMVA: prior daily dose of simvastatin in e.g. 10 mg units
7. For additional drugs:
   a. AMIODOSE: amiodarone dose in mg/day
   b. CARBDOSE: carbemazepine dose in mg/day
   c. PIOGDOSE: pioglitazone dose expressed as number of 15 mg doses per day
   d. DILTDOSE: diltiazem dose expressed as number of 120 mg doses per day
   e. METFDOSE: metformin dose expressed as number of 1000 mg doses per day
   f. PHENDOSE: phenyloin dose in mg/day
   g. RISPDOSE: risperidone dose in mg/day
   h. ROSIDOSE: rosiglitazone dose in mg/day
   i. VERADOSE: verapamil dose expressed as number of 120 mg doses per day

Body mass index (BMI) was chosen as a covariate of statin response because obese patients tend to consume a diet high in cholesterol. It was hypothesized that they would respond less well to statins because their hypercholesterolemia was more likely to be mediated by dietary indiscretion than excessive LDL synthesis. HbA1c was included because hypercholesterolemia is often found in patients with poorly controlled diabetes and because the United Kingdom Prospective Diabetes Study (UKPDS) found that Hba1c was an independent predictor of macrovascular events in type 2 diabetes. Metformin and the thiazolidinediones were chosen because they reduce LDL levels in diabetic patients through mechanisms that may or may not be affected by concurrent statin treatment. The remaining non-statin drugs were chosen because they are commonly used drugs that potentiate the effect of statins. They were selected by reviewing the official on-line VA pharmaceutical reference (Micromedex®) for major interactions mediated by impaired statin metabolism. A non-statin drug was considered “active” if the release date for the new statin prescription fell between the former's [release date] and [release date plus days supply]. The daily dose of the non-statin drug was calculated by the method described in Section B.

H. Assembling the predictor file—The patient identifier was used to link the new statin prescription type and dose, the preceding LDL above goal, and the type and dose of the previous statin (if any) with the patient's most recent HbA1c, most recent BMI, and the daily doses of 9 drugs with major statin interactions that were active at the time the new prescription was released.

I. Assembling the response file—The LDL on treatment file was also used to identify the last time that LDL was measured for a given dose and type of statin for each patient. This file contains records in which LDL was measured ≧42 days after the therapy was initiated. This task was accomplished by: 1) doing a 4-level ascending sort (by patient identifier, statin type, dose, and release date; and 2) grouping the sorted file by patient identifier, statin type, and dose and extracting the last record.

J. Linking predictor and response records—A specific dose and type of statin (say, 20 mg of simvastatin) was extracted from the predictor and response files. Extracted records from the predictor and response files were then linked by patient identifier. This process created a file in which the first prescription for simvastatin 20 mg/day, all covariates, and the last LDL under that treatment were linked. To develop a model for a specific conversion (say from lovastatin), these records were further selected for “previous statin=lovastatin”. This process was repeated to evaluate all available conversions within and between statin types.

K. Data analysis—Group differences in categorical variables were analyzed by chi-square. Group differences in continuous variables were examined by either the unpaired student's t-test or Mann-Whitney U test. Multivariate models were derived and validated on independent samples. For each model (e.g. simvastatin 10 mg daily), a microcomputer random number generated was used to assign cases to a derivation or validation set in a ratio of 1:1. The dependent variable was attainment of goal (LDL <100 mg/dl). The covariates were chosen because they were biologically plausible. Forward and backward stepping logistic regression was done on the derivation set to identify the factors most influential in predicting reaching goal. Step selections were based upon the maximal likelihood method with α<0.15 to enter and ≧0.15 to remove. The degree of improvement with each step was tested by improvement χ2. The goodness-of-fit for the final model was evaluated by the Hosmer-Lemeshow test because the number of covariate patterns was large in most cases. The ability of the model to discriminate between subjects reaching and not reaching goal was analyzed by calculating the area under the receiver operating characteristic (ROC) curve. Exploratory regressions started with an all-inclusive predictor set (and hence the smallest sample because cases with missing values are deleted). In subsequent steps, non-significant predictors were sequentially deleted from the list of candidate variables so that the final model tested only significant predictors on the largest possible sample. Validation was done on the test set. The logistic model was used to calculate a predicted probability of attaining goal for each member. The sample was then stratified into quintiles according to predicted probability. The proportion of subjects attaining goal across the categories was tested by χ2 analysis.

### II. Results

A. Source files—The following table shows the types of data and number of records used for this study:

| Source | Number |
|---|---|
| CV discharge diagnoses | 70,086 |
| CV problems on problem list | 159,053 |
| Diabetes medications | 462,425 |
| CABG | 1,020 |
| PTCA | 2,793 |
| LDL results | 363,889 |
| Statin prescriptions | 437,241 |

B. Identifying eligible subjects—Between FY 1996 and the end of Q1 of FY 2009, 33,484 patients were given the diagnosis of coronary artery disease (CAD) or its equivalent. Many patients did not have follow-up because they were referred to New Mexico Veterans Affairs Health Care System only for tertiary services or used the VA only for pharmacy benefits. Other patients were already at goal, not given a statin, treated with other lipid-lowering drugs, or on a disqualifying medication. The remaining 8,096 patients had an LDL ≧100 mg/dl and had statin therapy initiated or modified after their qualifying diagnosis. Many patients did not have a follow-up LDL because they died, transferred to the private sector, moved away, had an adverse event, were non-adherent, were lost to follow up, no longer had indications for aggressive treatment, or had the statin withdrawn. Some patients had their LDL measured ≧365 days before the statin was started, while others were on treatment for ≦42 days when the measurement was done. These patients were also removed from analysis.

C. Characteristics of patients attaining and not attaining goal—The remaining 5,964 subjects had their statin therapy modified 11,358 times because their LDL's were above goal. At last follow-up, 4,123 (69.1%) had an LDL <100 mg/dl, while 1,841 (30.9%) were still not at goal. The following table compares the clinical features of these two groups:

| | Goal | Not At Goal | |
|---|---|---|---|
| | (n = 4,123) | (n = 1,841) | P-value |
| Age (years) | 66.0 ± 10.0 | 63.9 ± 10.5 | <0.001 |
| % male | 69.9% | 55.3% | <0.001 |
| BMI (kg/M2) | 30.5 ± 5.8 | 30.3 ± 5.8 | NS |
| MPR—Medication Possession | 0.98 ± 0.22 | 0.96 ± 0.23 | NS |
| Ratio | | | |
| HbA1c (%) | 6.86 ± 1.60 | 6.92 ± 1.79 | NS |
| Pre-treatment LDL (mg/dL) | 130 ± 23 | 142 ± 28 | <0.001 |
| Last LDL (mg/dL) | 77 ± 15 | 123 ± 22 | <0.001 |
| Drop in LDL (mg/dL) | 53 ± 26 | 19 ± 31 | <0.001 |
| Decisions made | 1.79 ± 1.08 | 2.17 ± 1.20 | <0.001 |
| Follow-up (days) | 1,441 ± 1,081 | 1,086 ± 961 | <0.001 |

Patients reaching the LDL target were significantly younger, more likely to be male, and had a lower pre-treatment LDL. Moreover, the decrease in LDL was 3 times greater for those at versus not at goal, even though the former had significantly fewer dose titrations. Thus, the most important determinant of success is either the provider's choice of statin or the patient's biological response—not the intensity of follow-up. This observation reinforces the need for algorithms that guide statin selection and identify factors that modulate its response.

The 4,123 successfully treated patients reached their target in 413±523 days. The following table shows the time-to-coal for this cohort.

| Interval (days) | Patients |
|---|---|
| 0-99 | 1,143 |
| 100-199 | 855 |
| 200-299 | 446 |
| 300-399 | 384 |
| 400-499 | 236 |
| ≧500 | 1,059 |

2,268 (55.0%) of patients required ≧180 days; 1,402 (34.0%) required ≧365 days; and 708 (17.2%) required ≧730 days. Thus, nearly one-third of patients with an unequivocal indication for statin treatment never reached goal, and another third took over one year to be treated. This observation confirms that the current visit-based, provider-centric model of statin delivery has proved unsatisfactory.

Although the models discussed below pertain to lovastatin and simvastatin, it is contemplated the present invention is applicable to predicting the response of various other statins such as ceruvostatin, pitavastatin, pravastatin, fluvastatin, atorvastatin, osuvastatin, and rosuvastatin in treatment of a patient.

### III. Identifying Predictors of Lovastatin Response

A. Model specifications—The following tables show the models predicting the probability of goal for patients started on lovastatin at 10 mg, 20 mg, 40 mg and 80 mg per day. In the tables, SD=Standard Deviation, OR=Odds Ratio and CI=Confidence Interval.

Lovastatin 10 mg (n = 374)

| | Coeffi- | | | |
|---|---|---|---|---|
| Variable | cient | SD | OR | 95% CI |
| PLDL (per 10 mg/dl) | −0.4056 | 0.0686 | 0.667 | 0.582 to 0.763 |
| Age (per 10 years) | 0.3232 | 0.112 | 1.38 | 1.11 to 1.72 |
| Constant | 2.577 | 1.14 | 13.2 | 1.41 to 123 |

Hosmer-Lemeshow P = 0.985; ROC = 0.7217

Lovastatin 20 mg [A] (includes HbA1c; n = 595)

| | Coeffi- | | | |
|---|---|---|---|---|
| Variable | cient | SD | OR | 95% CI |
| HbA1c (%) | −0.1033 | 0.0545 | 0.902 | 0.810 to 1.00 |
| Diltiazem (per 120 mg/day) | 0.3654 | 0.233 | 1.44 | 0.912 to 2.28 |
| Metformin (per 1000 | 0.2609 | 0.129 | 1.30 | 1.01 to 1.67 |
| mg/day) | | | | |
| PLDL (per 10 mg/dl) | −0.2635 | 0.0442 | 0.768 | 0.704 to 0.838 |
| PLova (per 10 mg/day) | −0.4857 | 0.245 | 0.768 | 0.704 to 0.994 |
| Age (per 10 years) | 0.2085 | 0.0847 | 1.23 | 1.04 to 1.45 |
| Constant | 2.620 | 0.909 | 13.7 | 2.30 to 81.9 |

Hosmer-Lemeshow P = 0.801; ROC = 0.6819

Lovastatin 20 mg [B] (excludes HbA1c; n = 733)

| | Coeffi- | | | |
|---|---|---|---|---|
| Variable | cient | SD | OR | 95% CI |
| BMI | −0.02372 | 0.0146 | 0.977 | 0.949 to 1.00 |
| Pioglitazone (per 15 | 0.7210 | 0.468 | 2.06 | 0.820 to 5.16 |
| mg/day) | | | | |
| Diltiazem (per 120 | 0.5220 | 0.244 | 1.69 | 1.04 to 2.72 |
| mg/day) | | | | |
| Metformin (per 1000 | 0.2281 | 0.124 | 1.26 | 0.984 to 1.60 |
| mg/day) | | | | |
| PLDL (per 10 mg/dl) | −0.2943 | 0.0402 | 0.745 | 0.689 to 0.806 |
| PLova (per 10 mg/day) | −0.6130 | 0.221 | 0.542 | 0.351 to 0.835 |
| Age (per 10 years) | 0.1981 | 0.0775 | 1.22 | 1.05 to 1.42 |
| Constant | 3.096 | 0.943 | 22.1 | 3.47 to 141 |

Hosmer-Lemeshow P = 0.896; ROC = 0.6898

Lovastatin 40 mg (n = 764)

| | Coeffi- | | | |
|---|---|---|---|---|
| Variable | cient | SD | OR | 95% CI |
| Diltiazem (per 120 mg/day) | 0.3010 | 0.170 | 1.35 | 0.967 to 1.89 |
| Metformin (per 1000 mg/day) | 0.1983 | 0.113 | 1.22 | 0.976 to 1.52 |
| PLDL (per 10 mg/dl) | −0.2466 | 0.0413 | 0.781 | 0.721 to 0.848 |
| PLova (per 10 mg/day) | −0.2635 | 0.0798 | 0.768 | 0.657 to 0.899 |
| Age (per 10 years) | 0.2029 | 0.0764 | 1.22 | 1.05 to 1.42 |
| Constant | 1.865 | 0.782 | 6.46 | 1.39 to 30.0 |

Hosmer-Lemeshow P = 0.698; ROC = 0.6545

Lovastatin 80 (n = 309)

| | Coeffi- | | | |
|---|---|---|---|---|
| Variable | cient | SD | OR | 95% CI |
| PLDL (per 10 mg/dl) | −0.2270 | 0.0598 | 0.797 | 0.708 to 0.896 |
| PLova (per 10 mg/day) | −0.1420 | 0.0636 | 0.868 | 0.766 to 0.983 |
| Age (per 10 years) | 0.1886 | 0.122 | 1.21 | 0.949 to 1.54 |
| Constant | 2.038 | 1.14 | 7.67 | 0.812 to 72.5 |

Hosmer-Lemeshow P = 0.382; ROC = 0.6594

B. Comparison of odds ratios (OR) across models (bold and italicized indicates 95% CI does not include one)—The following table compares the adjusted ORs for the different lovastatin models:

| | | 20 mg | 20 mg | | |
|---|---|---|---|---|---|
| Variable | 10 mg | [A] | [B] | 40 mg | 80 mg |
| Age (per 10 years) | [symbol] | [symbol] | [symbol] | [symbol] | 1.21 |
| PLDL (per 10 mg/dl) | [symbol] | [symbol] | [symbol] | [symbol] | [symbol] |
| PLova (per 10 mg/day) | N/A | [symbol] | [symbol] | [symbol] | [symbol] |
| Diltiazem (per 120 | — | 1.44 | [symbol] | 1.35 | — |
| mg/day) | | | | | |
| Metformin (per 1000 | — | [symbol] | 1.26 | 1.22 | — |
| mg/day) | | | | | |
| Pioglitazone (per 15 | — | — | 2.06 | — | — |
| mg/day) | | | | | |
| BMI | — | — | [symbol] | — | — |
| HbA1c | — | [symbol] | — | — | — |

Note that age and pre-treatment LDL have a remarkably consistent effect across models. Increasing age is associated with a better statin response, while a high pre-treatment LDL confers a poor prognosis. Prior lovastatin treatment reduces the chances of reaching goal, an effect that is mitigated as higher doses are used. Finally, major drug interactions and a detrimental effect for a high BMI and high HbA1c were identified for the 20 mg and 40 mg models. The diltiazem effect was substantial (odds 70% greater for goal for every 120 mg per day) and probably mediated by inhibition of statin metabolism. However, a beneficial effect for metformin has not previously been described.

### IV. Validation of Lovastatin Models

A. Predicted probabilities—The predicted probability of attaining goal is given by the expression:

P(Goal)=e^{Σ}/(1+e^{Σ})

where Σ=Constant+β₁*variable₁+β₂*variable₂ . . . βₙ*variableₙ

B. Proportion of patients achieving goal in each quintile of predicted probability—Validation of the 80 mg model could not be done because of the small sample size. The predicted probability was calculated for each subject in the remaining validation sets using the models shown in Section VI. The validation set for each model was then stratified by predicted probability and the proportion attaining goal across categories tested by χ2 analysis:

| Predicted | 0-19% | 20-39% | 40-59% | 60-79% | 80-100% | Overall |
|---|---|---|---|---|---|---|
| 10 mg | 10.9% | 31.3% | 46.8% | 73.3% | (0) | 36.8% |
| | (55) | (80) | (77) | (30) | | |
| 20 mg [A] | 15.0% | 25.8% | 55.1% | 73.0% | 71.4% | 49.2% |
| | (40) | (151) | (314) | (122) | (7) | |
| 20 mg [B] | 12.7% | 33.0% | 53.6% | 75.0% | 64.7% | 49.3% |
| | (55) | (203) | (338) | (140) | (17) | |
| 40 mg | 11.1% | 31.2% | 50.7% | 70.7% | 100% | 49.3% |
| | (27) | (170) | (404) | (157) | (3) | |

Number in parentheses refers to patients assigned to each cell

Note that no lovastatin choice had more than a 50% chance of success. However, the models produce exquisite separation of patients with different likelihoods of reaching goal. They are also more effective in identifying those with a poor prognosis than a good prognosis. The reason is that most of the drugs potentiating statin response were not incorporated into models unless the sample size was ≧600.

### V. Identifying Predictors of Simvastatin Response

A. Model specifications—The following tables show the models predicting the probability of goal for patients started on simvastatin at 10 mg, 20 mg, 40 mg and 80 mg per day.

Simvastatin 10 mg [A] (no previous treatment; n = 172)

| | Coeffi- | | | |
|---|---|---|---|---|
| Variable | cient | SD | OR | 95% CI |
| Diltiazem (per 120 mg/day) | 0.5744 | 0.358 | 1.78 | 0.877 to 3.60 |
| Metformin (per 1000 mg/day) | 0.6102 | 0.361 | 1.84 | 0.902 to 3.76 |
| PLDL (per 10 mg/dl) | −0.2320 | 0.0704 | 0.793 | 0.690 to 0.911 |
| Age (per 10 years) | 0.5800 | 0.178 | 1.79 | 1.26 to 2.54 |
| Constant | −0.9924 | 1.47 | 0.371 | 0.0205 to 6.71 |

Hosmer-Lemeshow P = 0.568; ROC = 0.7236

Simvastatin 10 mg [B] (includes BMI; n = 136)

| | Coeffi- | | | |
|---|---|---|---|---|
| Variable | cient | SD | OR | 95% CI |
| BMI | 0.05125 | 0.0347 | 1.05 | 0.983 to 1.13 |
| PLDL (per 10 mg/dl) | −0.3430 | 0.0934 | 0.710 | 0.590 to 0.854 |
| PLova (per 10 mg/day) | −1.075 | 0.546 | 0.341 | 0.116 to 1.01 |
| PSimva (per 10 | −2.825 | 1.18 | 0.0593 | 0.00578 to 0.609 |
| mg/day) | | | | |
| Age (per 10 years) | 0.5869 | 0.205 | 1.80 | 1.20 to 2.70 |
| Constant | −0.6090 | 2.19 | 0.544 | 0.00718 to 41.2 |

Hosmer-Lemeshow P = 0.081; ROC = 0.7470

Simvastatin 10 mg [C] (excludes BMI; n = 219)

| | Coeffi- | | | |
|---|---|---|---|---|
| Variable | cient | SD | OR | 95% CI |
| Diltiazem (per 120 mg/day) | 0.5633 | 0.314 | 1.76 | 0.946 to 3.26 |
| Metformin (per 1000 mg/day) | 0.6817 | 0.348 | 1.98 | 0.995 to 3.93 |
| PLDL (per 10 mg/dl) | −0.2483 | 0.0667 | 0.780 | 0.684 to 0.890 |
| PLova (per 10 mg/day) | −0.9625 | 0.417 | 0.382 | 0.168 to 0.870 |
| PSimva (per 10 mg/day) | −1.902 | 0.893 | 0.149 | 0.0257 to 0.869 |
| Age (per 10 years) | 0.4918 | 0.163 | 1.64 | 1.19 to 2.26 |
| Constant | −0.2029 | 1.38 | 0.816 | 0.0542 to 12.3 |

Hosmer-Lemeshow P = 0.554; ROC = 0.7265

Simvastatin 20 mg (n = 374)

| | Coeffi- | | | |
|---|---|---|---|---|
| Variable | cient | SD | OR | 95% CI |
| PLDL (per 10 mg/dl) | −0.2783 | 0.0518 | 0.757 | 0.684 to 0.838 |
| PLova (per 10 mg/day) | −0.4836 | 0.164 | 0.617 | 0.447 to 0.851 |
| PSimva (per 10 mg/day) | −1.034 | 0.296 | 0.356 | 0.199 to 0.636 |
| Constant | 4.137 | 0.722 | 62.6 | 15.1 to 259 |

Hosmer-Lemeshow P = 0.677; ROC = 0.6902

Simvastatin 40 mg (n = 698)

| | Coeffi- | | | |
|---|---|---|---|---|
| Variable | cient | SD | OR | 95% CI |
| BMI | 0.03714 | 0.0153 | 1.04 | 1.01 to 1.07 |
| Diltiazem (per 120 mg/day) | 0.5284 | 0.353 | 1.70 | 0.848 to 3.39 |
| Metformin (per 1000 | 0.2732 | 0.120 | 1.31 | 1.04 to 1.66 |
| mg/day) | | | | |
| PLDL (per 10 mg/dl) | −0.2393 | 0.0397 | 0.787 | 0.728 to 0.851 |
| PLova (per 10 mg/day) | −0.08255 | 0.0455 | 0.921 | 0.842 to 1.01 |
| PSimva (per 10 mg/day) | −0.2385 | 0.110 | 0.788 | 0.635 to 0.978 |
| Age (per 10 years) | 0.2161 | 0.0896 | 1.24 | 1.04 to 1.48 |
| Constant | 0.8699 | 1.03 | 2.39 | 0.314 to 18.1 |

Hosmer-Lemeshow P = 0.605; ROC = 0.6663

Simvastatin 80 mg (n = 481)

| | Coeffi- | | | |
|---|---|---|---|---|
| Variable | cient | SD | OR | 95% CI |
| PLDL (per 10 mg/dl) | −0.1628 | 0.0410 | 0.850 | 0.784 to 0.921 |
| PSimva (per 10 mg/day) | −0.1204 | 0.0506 | 0.887 | 0.803 to 0.979 |
| Age (per 10 years) | 0.2409 | 0.103 | 1.27 | 1.04 to 1.56 |
| Constant | 1.375 | 0.866 | 3.95 | 0.722 to 21.7 |

Hosmer-Lemeshow P = 0.250; ROC = 0.6386

B. Comparison of OR's across models (bold and italicized indicates 95% CI)—The following table compares the adjusted OR's for variables in the different lovastatin models:

| | 10 mg | 10 mg | 10 mg | | | |
|---|---|---|---|---|---|---|
| | [A] | [B] | [C] | 20 mg | 40 mg | 80 mg |
| Age | [symbol] | [symbol] | [symbol] | — | [symbol] | [symbol] |
| (per 10 years) | | | | | | |
| PLDL | [symbol] | [symbol] | [symbol] | [symbol] | [symbol] | [symbol] |
| (per 10 mg/dl) | | | | | | |
| PLova | N/A | 0.341 | [symbol] | [symbol] | 0.921 | — |
| (per | | | | | | |
| 10 mg/day) | | | | | | |
| PSimva | N/A | [symbol] | [symbol] | [symbol] | [symbol] | [symbol] |
| (per | | | | | | |
| 10 mg/day) | | | | | | |
| Diltiazem | 1.78 | — | 1.76 | — | 1.70 | — |
| (per | | | | | | |
| 120 mg/day) | | | | | | |
| Metformin (per | 1.84 | — | 1.98 | — | [symbol] | — |
| 1000 mg/day) | | | | | | |
| BMI | | 1.05 | — | — | [symbol] | — |

Again, the beneficial effect of age and detrimental effect of a high prior LDL are remarkably consistent across models. Note that, as in the lovastatin models, the poor prognosis associated with prior treatment with either lovastatin or simvastatin attenuates as higher doses of simvastatin are used. Diltiazem and metformin are again identified as potentiators of statin effect in the model with the greatest sample size (40 mg).

### VI. Validation of Simvastatin Models

The simvastatin models were validated in the same manner as the lovastatin models:

| Predicted | 0-19% | 20-39% | 40-59% | 60-79% | 80-100% | Overall |
|---|---|---|---|---|---|---|
| 10 mg [A] | 8.3% | 29.7% | 62.1% | 68.6% | 75.0% | 48.7% |
| | (12) | (37) | (29) | (35) | (4) | |
| 10 mg [B] | 10.0% | 26.3% | 58.8% | 70.6% | 100% | 52.1% |
| | (10) | (19) | (17) | (17) | (10) | |
| 10 mg [C] | 11.8% | 31.8% | 60.0% | 74.2% | 75.0% | 47.6% |
| | (17) | (44) | (30) | (31) | (4) | |
| 20 mg [C] | 10.5% | 35.1% | 47.2% | 65.3% | (0) | 50.1% |
| | (19) | (74) | (144) | (150) | | |
| 40 mg | 20.0% | 38.6% | 54.6% | 67.0% | 65.0% | 57.4% |
| | (15) | (88) | (269) | (288) | (20) | |
| 80 mg | (0) | 41.7% | 41.9% | 72.8% | 70.8% | 61.9% |
| | | (12) | (124) | (323) | (24) | |

Number in parentheses refers to the patients assigned to each cell

Note that the chances of success were better with simvastatin than lovastatin but did not exceed 62%. However, the simvastatin models identified large numbers of subjects with such a low probability that they should probably not have been treated.

### VII. Models Containing Common Data Elements

For now, predicted probabilities of goal should be compared for different types and doses of statins only if their models contain the same data elements. There are 2 reasons for this precaution. Certain variables (e.g. HbA1c) are more likely to be measured in patient subsets (diabetes) whose statin response could differ substantially from the general population. As a result, algorithms containing and not containing HbA1c predict response rates in different populations. Other variables were incorporated into models because a larger sample size allowed the identification of more predictors. For example, metformin was included in the simvastatin 40 mg model (n=698) but not in the simvastatin 20 mg (n=374) or simvastatin 80 mg (n=471) models. In the 40 mg model, the odds of reaching goal are 30% higher if the patient is taking metformin. If the 20 mg and 80 mg models are under-specified with respect to metformin, the predicted probabilities of achieving goal will be under-estimated. For example, the probability of goal could increase dramatically from 20 mg to 40 mg but then decrease from 40 mg to 80 mg. Until the effect of metformin can be rigorously assessed in all models, it should not be included in any.

Parameters for common models were obtained by fitting the same set of variables to each derivation set. The purpose was to estimate the coefficients for all common data elements even if they are not statistically significant. Trends in the predicted probability of success across preparations could then be compared. From tables III-B and V-B, it is clear that the common models should contain age, PLDL, PLova, and PSimva. It is unclear if they should also contain BMI, diltiazem, and metformin. Accordingly, 3 models containing common data elements were compared to the models from stepwise regression:
A. Model 1: age, PLDL, PLova, and PSimva
B. Model 2: age, PLDL, Plova, PSimva, and BMI
C. Model 3: age, PLDL, PLova, PSimva, BMI, Diltdose, and Metfdose

Three strategies were used to test which of the 3 fitted models provided the best description of the data: Hosmer-Lemeshow P-value on the derivation set; ROC area on the derivation set; and χ2 statistic on the validation set. These models were also compared to the ones derived from stepwise regression using all candidate variables.

Goodness-of-fit (bold and italicized indicates
highest value across 4 models)

| | Stepwise | Model 1 | Model 2 | Model 3 |
|---|---|---|---|---|
| Lova10 | [symbol] | [symbol] | 0.882 | 0.259 |
| Lova20 | 0.896 | 0.783 | 0.774 | [symbol] |
| Lova40 | 0.698 | [symbol] | 0.902 | 0.129 |
| Lova80 | [symbol] | [symbol] | 0.164 | 0.146 |
| Simva10 | 0.081 | [symbol] | 0.081 | 0.103 |
| Simva20 | 0.677 | 0.234 | [symbol] | 0.461 |
| Simva40 | [symbol] | 0.370 | 0.571 | [symbol] |
| Simva80 | 0.250 | 0.147 | [symbol] | 0.405 |

ROC areas (bold and italicized indicates
highest value across 4 models)

| | Stepwise | Model 1 | Model 2 | Model 3 |
|---|---|---|---|---|
| Lova10 | [symbol] | [symbol] | 0.7157 | 0.7201 |
| Lova20 | [symbol] | 0.6779 | 0.6804 | 0.6862 |
| Lova40 | 0.6545 | 0.6459 | 0.6498 | [symbol] |
| Lova80 | 0.6594 | 0.6594 | 0.6593 | [symbol] |
| Simva10 | 0.7470 | 0.7014 | 0.7470 | [symbol] |
| Simva20 | 0.6902 | [symbol] | 0.6765 | 0.6750 |
| Simva40 | 0.6663 | 0.6464 | 0.6568 | [symbol] |
| Simva80 | 0.6386 | 0.6381 | 0.6396 | [symbol] |

χ2 statistic on validation set (bold and italicized
indicates highest value across 4 models)

| | Stepwise | Model 1 | Model 2 | Model 3 |
|---|---|---|---|---|
| Lova10 | [symbol] | [symbol] | 36.602 | 36.795 |
| Lova20 | 92.051 | [symbol] | 102.664 | 99.293 |
| Lova40 | 70.283 | 66.954 | 61.954 | [symbol] |
| Lova80 | — | — | — | — |
| Simva10 | [symbol] | 20.601 | [symbol] | 22.594 |
| Simva20 | 32.931 | [symbol] | 25.698 | 24.440 |
| Simva40 | 33.433 | [symbol] | 38.329 | 33.433 |
| Simva80 | [symbol] | 30.160 | 25.779 | 17.498 |

This analysis shows that Model 1 is equivalent or superior to the other models in terms of goodness-of-fit and performance on the validation set. Model 3 provides the best ROC area. For this reason, model 1 was tested and validated for all doses of lovastatin and simvastatin.

### VIII. Model 1: age, PLDL, PLova, and PSimva

A. Model Specifications

Lovastatin 10 mg

| | Coeffi- | | | |
|---|---|---|---|---|
| Variable | cient | SD | OR | 95% CI |
| Age (per 10 years) | 0.3232 | 0.112 | 1.38 | 1.11 to 1.72 |
| PLDL (per 10 mg/dl) | −0.4056 | 0.0686 | 0.667 | 0.582 to 0.763 |
| PLova (per 10 mg/day) | N/A | N/A | N/A | N/A |
| PSimva (per 10 mg/day) | N/A | N/A | N/A | N/A |
| Constant | 2.577 | 1.14 | 13.2 | 1.41 to 123 |

Hosmer-Lemeshow P = 0.985; ROC = 0.7217

Lovastatin 20 mg

| | Coeffi- | | | |
|---|---|---|---|---|
| Variable | cient | SD | OR | 95% CI |
| Age (per 10 years) | 0.2267 | 0.0715 | 1.25 | 1.09 to 1.44 |
| PLDL (per 10 mg/dl) | −0.2778 | 0.0375 | 0.757 | 0.704 to 0.815 |
| PLova (per 10 mg/day) | −0.5218 | 0.212 | 0.593 | 0.392 to 0.899 |
| PSimva (per 10 mg/day) | N/A | N/A | N/A | N/A |
| Constant | 2.026 | 0.693 | 7.58 | 1.95 to 29.5 |

Hosmer-Lemeshow P = 0.783; ROC = 0.6779

Lovastatin 40 mg

| | Coeffi- | | | |
|---|---|---|---|---|
| Variable | cient | SD | OR | 95% CI |
| Age (per 10 years) | 0.1800 | 0.0752 | 1.20 | 1.03 to 1.39 |
| PLDL (per 10 mg/dl) | −0.2504 | 0.0411 | 0.778 | 0.718 to 0.844 |
| PLova (per 10 mg/day) | −0.2614 | 0.0795 | 0.770 | 0.659 to 0.900 |
| PSimva (per 10 mg/day) | N/A | N/A | N/A | N/A |
| Constant | 2.153 | 0.766 | 8.61 | 1.91 to 38.7 |

Hosmer-Lemeshow P = 0.954; ROC = 0.6459

Lovastatin 80 mg

| | Coeffi- | | | |
|---|---|---|---|---|
| Variable | cient | SD | OR | 95% CI |
| Age (per 10 years) | 0.1886 | 0.122 | 1.21 | 0.949 to 1.54 |
| PLDL (per 10 mg/dl) | −0.2270 | 0.0598 | 0.797 | 0.708 to 0.896 |
| PLova (per 10 mg/day) | −0.1420 | 0.0636 | 0.868 | 0.766 to 0.983 |
| PSimva (per 10 mg/day) | N/A | N/A | N/A | N/A |
| Constant | 2.038 | 1.14 | 7.67 | 0.812 o 72.5 |

Hosmer-Lemeshow P = 0.382; ROC = 0.6594

Simvastatin 10 mg

| | Coeffi- | | | |
|---|---|---|---|---|
| Variable | cient | SD | OR | 95% CI |
| Age (per 10 years) | 0.4482 | 0.157 | 1.57 | 1.15 to 2.13 |
| PLDL (per 10 mg/dl) | −0.2362 | 0.0646 | 0.790 | 0.695 to 0.897 |
| PLova (per 10 mg/day) | −0.8355 | 0.391 | 0.434 | 0.200 to 0.938 |
| PSimva (per 10 mg/day) | −2.041 | 0.881 | 0.130 | 0.0229 to 0.738 |
| Constant | 0.09086 | 1.31 | 1.10 | 0.0828 to 14.5 |

Hosmer-Lemeshow P = 0.689; ROC = 0.7014

Simvastatin 20 mg

| | Coeffi- | | | |
|---|---|---|---|---|
| Variable | cient | SD | OR | 95% CI |
| Age (per 10 years) | 0.04368 | 0.113 | 1.04 | 0.836 to 1.31 |
| PLDL (per 10 mg/dl) | −0.2765 | 0.0519 | 0.758 | 0.685 to 0.840 |
| PLova (per 10 mg/day) | −0.4866 | 0.164 | 0.615 | 0.445 to 0.849 |
| PSimva (per 10 mg/day) | −1.035 | 0.296 | 0.355 | 0.199 to 0.636 |
| Constant | 3.830 | 1.07 | 46.0 | 5.59 to 379 |

Hosmer-Lemeshow P = 0.234; ROC = 0.6915

Simvastatin 40 mg

| | Coeffi- | | | |
|---|---|---|---|---|
| Variable | cient | SD | OR | 95% CI |
| Age (per 10 years) | 0.1327 | 0.0832 | 1.14 | 0.970 to 1.34 |
| PLDL (per 10 mg/dl) | −0.2446 | 0.0376 | 0.783 | 0.727 to 0.843 |
| PLova (per 10 mg/day) | −0.07449 | 0.0441 | 0.928 | 0.851 to 1.01 |
| PSimva (per 10 mg/day) | −0.2603 | 0.103 | 0.771 | 0.630 to 0.944 |
| Constant | 2.724 | 0.770 | 15.2 | 3.36 to 69.1 |

Hosmer-Lemeshow P = 0.370; ROC = 0.6464

Simvastatin 80 mg

| | Coeffi- | | | |
|---|---|---|---|---|
| Variable | cient | SD | OR | 95% CI |
| Age (per 10 years) | 0.2334 | 0.103 | 1.26 | 1.03 to 1.55 |
| PLDL (per 10 mg/dl) | −0.1599 | 0.0411 | 0.852 | 0.786 to 0.924 |
| PLova (per 10 mg/day) | 0.04375 | 0.0527 | 1.04 | 0.942 to 1.16 |
| PSimva (per 10 mg/day) | −0.1049 | 0.0536 | 0.900 | 0.810 to 1.00 |
| Constant | 1.325 | 0.868 | 3.76 | 0.683 to 20.7 |

Hosmer-Lemeshow P = 0.147; ROC = 0.6381

B. Validation of Model 1—The following table shows that model 1 produces consistent separation of patients with different prognoses regardless of drug type or dose. Again, the models are better at identifying patients with a poor compared to good prognosis.

| Predicted | 0-19% | 20-39% | 40-59% | 60-79% | 80-100% | Overall |
|---|---|---|---|---|---|---|
| Lova10 | 10.9% | 31.3% | 46.8% | 73.3% | (0) | 36.8% |
| | (55) | (80) | (77) | (30) | | (242) |
| Lova20 | 8.5% | 29.0% | 54.4% | 74.6% | (0) | 47.4% |
| | (59) | (221) | (406) | (130) | | (816) |
| Lova40 | 11.1% | 33.3% | 49.1% | 73.2% | (0) | 49.3% |
| | (27) | (159) | (422) | (153) | | (761) |
| Lova80 | None | None | None | None | None | None |
| Simva10 | 14.3% | 31.8% | 57.6% | 69.7% | 100% | 47.6% |
| | (14) | (44) | (33) | (33) | (2) | (126) |
| Simva20 | 11.1% | 32.9% | 46.8% | 66.2% | (0) | 49.9% |
| | (18) | (73) | (141) | (145) | | (377) |
| Simva40 | 36.4% | 30.4% | 54.2% | 67.2% | 0% | 57.0% |
| | (11) | (79) | (306) | (314) | (1) | (711) |
| Simva80 | (0) | 42.7% | 42.7% | 68.9% | 74.3% | 61.9% |
| | | (12) | (124) | (312) | (35) | (483) |

P < 0.001 for all models

C. Comparing Or for Model 1 Across Doses

Lovastatin

| Variable | 10 mg | 20 mg | 40 mg | 80 mg |
|---|---|---|---|---|
| Age (per 10 years) | 1.38 | 1.25 | 1.20 | 1.21 |
| PLDL (per 10 mg/dl) | 0.667 | 0.757 | 0.778 | 0.797 |
| PLova | N/A | 0.593 | 0.770 | 0.868 |
| PSimva | N/A | N/A | N/A | N/A |

Simvastatin

| Variable | 10 mg | 20 mg | 40 mg | 80 mg |
|---|---|---|---|---|
| Age (per 10 years) | 1.57 | 1.04 | 1.14 | 1.26 |
| PLDL (per 10 mg/dl) | 0.790 | 0.758 | 0.783 | 0.852 |
| PLova | 0.434 | 0.615 | 0.928 | 1.04 |
| PSimva | 0.130 | 0.355 | 0.771 | 0.900 |

This analysis shows that age has an inconsistent effect across lovastatin and simvastatin doses. However, the detrimental effect of a high PLDL, a high current dose of lovastatin, and a high current dose of simvastatin attenuates as the dose of the next medication increases.

### IX. Basic Program for Calculating Predicted Probabilities of Attaining Goal Using Model 1

The following is the program that uses a patient file (PTDATA) containing age, prior LDL level, prior lovastatin dose, and prior simvastatin dose to estimate the probability of success for all lovastatin and simvastatin choices:

### US PTDATA

### SA OUTPUT

### 100 LET AGE10=AGE/10

### 110 LET PLDL10=PLDL/10

### 120 LET PLOVA10=PLOVA/10

### 130 LET PSIMVA10=PSIMVA/10

### 200 LET SUML10=0.3232*AGE10-0.4056*PLDL10+2.577

### 210 LET EXPL10=EXP(SUML10)

### 220 LET PROBL10=EXPL10/(1+EXPL10)

### 300 LET SUML20=0.2267*AGE10−0.2778*PLDL10−0.5218*PLOVA10+2.026

### 310 LET EXPL20=EXP(SUML20)

### 320 LET PROBL20=EXPL20/(1+EXPL20)

### 400 LET SUML40=0.1800*AGE10−0.2504*PLDL10−0.2614*PLOVA10+2.153

### 410 LET EXPL40=EXP(SUML40)

### 420 LET PROBL40=EXPL40/(1+EXPL40)

### 500 LET SUML80=0.1886*AGE10−0.2270*PLDL10−0.1420*PLOVA10+2.038

### 510 LET EXPL80=EXP(SUML80)

### 520 LET PROBL80=EXPL80/(1+EXPL80)

### 600 LET SUMS10=0.4482*AGE10−0.2362*PLDL10−0.8355*PLOVA10−2.041*PSIMVA10+0.09086

### 610 LET EXPS10=EXP(SUMS10)

### 620 LET PROBS10=EXPS10/(1+EXPS10)

### 700 LET SUMS20=0.04368*AGE10−0.2765*PLDL10−0.4866*PLOVA10−1.035*PSIMVA10+3.830

### 710 LET EXPS20=EXP(SUMS20)

### 720 LET PROBS20=EXPS20/(1+EXPS20)

### 800 LET SUMS40=0.1327*AGE10−0.2446*PLDL10−0.07449*PLOVA10−0.2603*PSIMVA10+2.724

### 810 LET EXPS40=EXP(SUMS40)

### 820 LET PROBS40=EXPS40/(1+EXPS40)

### 900 LET SUMS80=0.2334*AGE10−0.1599*PLDL10+0.04375*PLOVA10-0.1049*PSIMVA10+1.325

### 910 LET EXPS80=EXP(SUMS80)

### 920 LET PROBS80=EXPS80/(1+EXPS80)

### RU

### X. Probability of Reaching Goal for 68 Y/O Male with PLDL 143 on Lovastatin 20 MG/DL

The following matrix of probabilities was created by running the program in Section IX for a hypothetical patient.

| | Lovastatin | Simvastatin |
|---|---|---|
| 10 mg | N/A | N/A |
| 20 mg | Current Rx | 0.310 |
| 40 mg | 0.326 | 0.495 |
| 80 mg | 0.448 | 0.671 |

This analysis shows that lovastatin 40 mg (the currently recommended choice) or switching to simvastatin 20 mg is associated with the same low probability of reaching goal and should not be attempted. In fact, a more aggressive approach (lovastatin 80 mg or simvastatin 40 mg) is still more likely to fail than succeed. The most appropriate choice is simvastatin 80 mg. This example illustrates the value of a validated, explicit, evidence-based approach to statin selection rather than current “recommendations”.

### XI. Discussion

The statins are remarkably effective in reducing cardiovascular mortality, well-tolerated, and now highly affordable. Despite these favorable attributes, a substantial proportion of patients eligible for treatment never receive it, and nearly half of those treated do not reach their target LDL. The reason for these poor outcomes is unknown. The latter phenomenon has been attributed to “clinical inertia” even though there is little evidence that health care providers lack the initiative to treat hypercholesterolemia. In fact, a study was recently completed showing that the opposite might be true. The factors were analyzed affecting the decision to intensify statin treatment in 2,699 patients making 3,768 visits to their primary care providers. No action was taken in 67.1% of visits where the LDL was above the ATP target. Hierarchical regression identified barriers at the institutional, provider, patient, and visit level. For example, after controlling for covariates, a missed opportunity was more common at rural than urban sites, more frequent among non-academic practitioners than medical residents, more likely for women than men, and more frequent when the change required a non-formulary preparation. These observations suggest that provider incentives are unlikely to succeed unless economic, cultural, or administrative barriers are addressed. The present study provides further indictment of the visit-based, provider-centric approach to statin treatment. Over the past 12 years, 31% of nearly 6,000 patients with CAD or its equivalent never reached their target LDL after treatment was intensified. Among those not at target, statin therapy was changed only twice in over 3 years of follow-up. Among those who reached their targets, the time to goal was ≧1 year for 34% of patients and ≧2 years for 17%. Finally, no lovastatin choice had more than a 50% success rate, and no simvastatin choice except 80 mg daily had more than a 60% chance of success. These observations suggest that there are limited opportunities for statin adjustments in a primary care setting, that barriers can be a significant deterrent, and that providers need decision-support to make better treatment choices. Much more attention should be devoted to alternative delivery systems that are more accessible, responsive to patient needs, and driven by a rigorous evaluation of the factors that affect treatment response.

In this study, patients reaching their targets had a much greater drop in their LDL even though they received significantly fewer dose adjustments. This observation suggested that statin selection and biologic response were more important determinants of success than the intensity of follow-up. For this reason, multivariate models were developed predicting statin response based upon variables that should have an impact on statin effect or metabolism. It was found that, for differing models, success was more likely in older patients and those who had better glycemic control, who were treated with certain other medications, who had a lower pre-treatment LDL, and who had not previously treated with higher doses of statins. All models were validated in independent samples.

Several studies have shown that statin response improves with age (12-16). The reasons are unknown but may include age-related changes in body size or composition, decreases in hepatic cholesterol synthesis, decreases in statin metabolism, or changes in behaviors such as medication adherence or diet. However, the age effect found in this study is much larger than previously reported. For example, for simvastatin 10 mg model, the odds of success increased by 80% for every 10 years. This effect is clinically significant and should be considered when treatment is intensified.

UKPDS showed that glycemic control is an important predictor of macrovascular complications in type 2 diabetes. Moreover, hypercholesterolemia is a well-known consequence of poor metabolic control. Our study extends these observations by showing that, for the simvastatin 20 mg model, the response to statins in better among those with lower HbA1c. It was also found that metformin had a synergistic effect with statins in 2 models—a finding that has not previously been reported. The odds ratio for concurrent metformin treatment was 1.3 for every 1,000 mg daily.

Lovastatin and simvastatin are metabolized by hepatic cytochrome CYP3A4—a mechanism shared by other drugs commonly used in cardiac patients. As a result, concurrent treatment with diltiazem, verapamil, or amiodarone can lead to elevated statin levels. The rationale for reducing statin doses in these patients is that the risk of hepatotoxicity and rhabdomyolysis is increased. Our study provides another justification for using lower doses—improved statin effectiveness. For the lovastatin 20 mg model, this effect was statistically significant and clinically important. For very 120 mg/day increase in diltiazem dose, the odds of reaching goal increased by 70%. Again, to our knowledge, this finding has not previously been reported.

Finally, our study suggests that the usual approach to statin titration is fundamentally flawed. Statin potency is conventionally expressed as the percent reduction in LDL observed in statin-naïve subjects treated in clinical trials. Reference tables have been constructed showing the expected LDL reduction for each dose and type of statin. Providers using these tables to modify statin therapy make two assumptions: 1) that the factors affecting statin response in routine practice are clinically unimportant; and 2) that patients failing one dose or type of statin have the same response to a new preparation as a statin-naïve subject. Our findings show that neither of these assumptions is justified. For example, elderly patients are not recruited to statin trials because the benefits of treatment are unclear. Moreover, patients on drugs with significant statin interactions may not be recruited because of the higher risk of toxicity. If the selection of subjects for trials is biased by these criteria, our study suggests that an elderly patient on those drugs would have a greater response to a given statin than predicted by trials data. Moreover, patients failing a previous statin regimen have a lower probability of reaching goal with a new preparation than untreated patients. This observation suggests that a patient requiring modification of statin therapy could have a response much less than that predicted from clinical trials on previously untreated patients. For example, for simvastatin 20 model, the odds of reaching goal are reduced by 70% for every 10 mg/day in the previous simvastatin dose.

In summary, the outcomes of visit-based treatment are poor, time to goal is unreasonable, opportunities for treatment are offset by barriers at multiple levels, individual choices made by practitioners have a low probability of success, multiple factors affect statin response, and their interactions are too complex to be assessed in a subjective manner. These observations suggest that a fundamental change is required in the way that statins are prescribed. The most appropriate alternative is a computerized set of evidence-based decision rules based upon all relevant factors coupled with automatic approval of non-formulary preparations if they are the only ones likely to succeed, dose titrations outside of the clinic setting, and even structured self-titration for selected patients. Fortunately, our prediction rules show a great deal of promise for guiding treatment and achieving goal in the most efficient manner.

Separate models have been developed in the present invention for lovastatin at doses of 10 mg, 20 mg, 40 mg, and 80 mg daily. Age, prior LDL level, and prior lovastatin dose were identified as predictors for models. Diltiazem dose, metformin dose, BMI, and HbA1c were incorporated into models using the largest numbers of patients—suggesting that they might be included in all models when additional populations are studied. When applied to an independent sample, the models produced remarkable separation of groups with different likelihoods of reaching goal (LDL <100 mg/dl) (from 11% to 75%). Models for simvastatin have also been developed in the present invention at the same doses. Age, prior LDL level, prior lovastatin dose, and prior simvastatin dose were identified as predictors for all models. The other variables were again selected for models using greater sample sizes. When applied to separate validation sets, the models again produced a clean separation of patients with different success rates (from 8% to 100%). It is contemplated that models can be developed according to the present invention for ceruvostatin, pitavastatin, pravastatin, fluvastatin, atorvastatin, osuvastatin, and rosuvastatin.

### XII. Algorithm Applications

Because of their complexity, the algorithms should be used as the basis for computerized decision support—either as a stand-alone office application, located on an Internet website, or embedded in an electronic medical record (EMR). Either the patient or provider can enter the patient's relevant clinical data. Alternatively, the inputs can be read automatically from different parts of the EMR. For patients starting therapy, the output would consist of a table listing the predicted probabilities of achieving target LDL for each dose of all available statins. For patients already under treatment, the output would list the probability of goal for higher doses of the same statin or equivalent/higher doses of more potent statins. The provider can then choose the least costly preparation and dose that have a reasonable probability of success.

### XIII. References

1. Austin P C, Mamdani M M, Juurlink D N. How many “Me-Too” drugs are enough? The case of physician preference for specific statins. Ann Pharmacother 2006; 40(6):1047-51.
2. Buusman A, Kragstrup J, Andersen M. General practitioners choose within a narrow range of drugs when initiating new treatments: a cohort study of cardiovascular drug formularies. Eur J Clin Pharmacol 2005; 61(9):651-6.
3. Mamdani M M, Tu K, Austin P C, Alter D A. Influence of socio-economic status on drug selection for the elderly in Canada. Ann Pharmacother 2002; 36(5):804-8.
4. Bradford W D, Kleit A N, Nietert P J, Ornstein S. Effects of direct-to-consumer advertising of hydroxymethylgutaryl co-enzyme a reductase inhibitors on attainment of LDL-C goals. Clin Ther 2006; 28(12):2105-18.
5. Cox E R, Kulkami A, Henderson R. Impact of patient and plan design factors on switching to preferred statin therapy. Ann Pharmacother 2007; 41(12):1946-53.
6. Hess G, Sanders K N, Hill J, Liu L Z. Therapeutic dose assessment of switching from atorvastatin to simvastatin. Am J Manag Care 2007; 13(Suppl 3); S80-5.
7. Thiebaud P, Patel B V, Nichol M B, Berenbeim D M. The effect of switching on compliance and persistence: the case of statin treatment. Am J Manag Care 2005; 11(11):670-4.
8. Weymiller A J, Montori V M, Jones L A, Gafni A, Guyatt G H, Bryant S C, Christianson T J, Mullan R J, Smith S A. Helping patients with type 2 diabetes mellitus make therapeutic decisions: a statin choice randomization trial. Arch Intern Med 2007; 167(10):1076-82.
9. Parris E S, Lawrence D B, Mohn L A, Long L B. Adherence to statin therapy and LDL cholesterol goal attainment by patients with diabetes and dyslipidemia. Diabetes Care 2005; 28(3):595-9.
10. Grant R W, Buse J B, Meigs J B for the University Health System Consortium (UHC) Diabetes Benchmarking Project Team. Quality of diabetes care in U.S. academic medical centers: low rates of medical regimen change. Diabetes Care 2005; 28:337-342.
11. Kennedy A G, MacLean C D, Littenberg B, Ades P A, Pinckney R G. The challenge of achieving national cholesterol goals in patients with diabetes. Diabetes Care 2005; 28:1029-34.
12. Simon J A, Lin F, Hulley S B, Blanche P J, Waters D, Shiboski S, Rotter J I, Nickerson D A, Yang H, Saad M, Krauss R M. Phenotypic predictors of response to Simvastatin therapy among African-Americans and Caucasians: the Cholesterol and Pharmacogenetics (CAP) Study. Am J Cardiol 2006; 97:843-50.
13. Shear C L, Franklin F A, Stinnett S, Hurley D P, Bradford R H, Chremos A N, Nash D T, Langendorfer A. Expanded Clinical Evaluation of Lovastatin (EXCEL) study results: effect of patient characteristics on lovastatin-induced changes in plasma concentrations of lipids and lipoproteins. Circulation 1992; 85:1293-1303.
14. Hunt D, Young P, Simes J, Hague W, Mann S, Owensby D, Lane G, Tonkin A. Benefits of pravastatin on cardiovascular events and mortality in older patients with coronary disease are equal to or exceed those seen in younger patients: results from the LIPID trial. Ann Intern Med 2001; 134:931-40.
15. Kannel W B, D'Agostino R B, Stepanians M, D'Agostino L C. Efficacy and tolerability of lovastatin in a six-month study: analysis by gender, age, and hypertensive status. Am J Cardiol 1990; 66(suppl):1B-10B.
16. Lewis S J, Moye L A, Sacks F M, Johnstone D E, Timmis G, Mitchell J, Limacher M, Kell S, Glasser S P, Grant J, et al. Effect of pravastatin on cardiovascular events in older patients with myocardial infarction and cholesterol levels in the average range: results of the Cholesterol and Recurrent Events (CARE) trial. Ann Intern Med 1998; 129:681-9.
17. Ose L, Luurila O, Eriksson J, Olsson A, Lithell H, Widgren B. Efficacy and safety of cerivastatin, 0.2 mg and 0.4 mg, in patients with primary hypercholesterolaemia: a multinational, randomized, double-blind study. Curr Med Res Opin 1999; 15:228-40.
18. Stagnitti M N. Trends in statins utilization and expenditures for the U.S. civilian noninstitutionalized population, 2000 and 2005. Statistical brief 205# from the Medical Expenditure Panel Survey. Agency for Healthcare Research and Quality, May 2008.
19. Tinetti M E. Over-the-counter sales of statins and other drugs for asymptomatic conditions. N Engl J Med 358; 25:2728-32.
20. Choudhry N K. Over-the-counter statins. Ann Intern Med 2005; 142:910-3.
21. Gotto A M Jr. Is it appropriate to make statins available over the counter? Circulation 2006; 114:1310-4.
22. Strom B L. Statins and over-the-counter availability. N Engl J Med 2005; 352:1403-5.
23. Melin J M, Struble W E, Tipping R W, Reynolds J M, Vassil T C, Levy S J, et al. A Consumer Use Study of Over-the-Counter lovastatin (CUSTOM). Am J Cardiol 2004; 94:1243-8.
24. Brass E P. Consumer behavior in the setting of over-the-counter statin availability; lesions from the consumer use study of OTC Mevacor. Am J Cardiol 2004; 94:22 F-29F.
25. Brass E P, Allen S E, Melin J M. Potential impact on cardiovascular public health of over-the-counter statin availability. Am J Cardiol 2006; 97:851-6.
26. Hu L. FDA briefing document: SELECT study NDA 21-213 proposing over-the-counter Mevacor (lovastatin) 20 mg. Accessed May 30, 2008 at http://www.fda.gov/ohrms/dockets/ac/07/briefing/2007-4331b1-01-FDA.pdf).

### Example 2

Statin Manager represents the convergence of two rapidly-evolving strategies—self management and Internet-based treatment. The underlying concepts have been validated for other conditions. This proposal integrates these approaches and extends their application to a substantial public health problem.

### I. System Components

Stalin Manager will reside on a secured website on the internet. The system has a lock-out feature to prevent premature use of the algorithms. Based upon information provided at registration, the site generates a target LDL unless the primary care provider overrides this option (see Section V—Treatment Targets). The treatment algorithm is based on MicroMedex (an on-line pharmacy reference) and is designed to emulate best practices. A Dose Titration Module (Section VI) guides the patients through a series of questions, laboratory tests, and medication changes until they are either withdrawn from the protocol or achieve their target LDL. The site automatically identifies the preparation, dose, and timing for each step based upon the allowable statins, their potency, and their time to peak effect (see Section XI—Treatment Rules). Patients at goal are then transferred to the Drug Maintenance Module for a periodic evaluation of LDL and drug side effects (see Section VII). Over the course of years, patients may alternate between the two modules to keep their LDL at goal. Throughout this process, a nurse monitors patient progress by reviewing adherence to the protocol, laboratory results, and data provided during the sessions (see Section XIII—Adherence and Safety Monitoring). For each change of medication, the site generates the dates for the next laboratory tests and session, sends out e-mail reminders to patients before those dates, and sends e-mails and/or letters to the primary care providers advising them of the changes (see Section XII—Date Rules). The system can either be used as a stand-alone program by individual practitioners or integrated into health care systems. In the latter case, the algorithm will be based upon formulary statins, laboratory data will automatically be retrieved, and progress notes will be written to the electronic medical record.

### II. Eligible Patients

Patients must meet the following prerequisites:
1. Proficiency in the English language
2. High school diploma (or equivalent)
3. No diagnosis of depression or cognitive dysfunction
4. Experience using the internet for other tasks (e-mail, banking, shopping, trip planning, education, work).

Elevation of liver function tests occurs in 0.5%-2.0% of patients on statins. Severe myopathy has been reported in 0.08% to 0.09% of patients on lovastatin, simvastatin, and pravastatin. Certain conditions increase the risk of these complications and are contraindications to the use of the System (20):
1. Age ≧80 years
2. BMI <22
3. Multi-system disease
4. Chronic renal insufficiency
5. Untreated hypothyroidism
6. Peri-operative patients
7. Alcoholism or substance abuse
8. Active liver disease.

### III. Provider Inputs

Primary care providers must authorize the use of the protocol for their patients and select the statins to be used. Statin Manager checks the patient's medication profile against an array of herbal and prescription medications that increase the risk of statin toxicity. It sends a warning to the referring provider when such an interaction is encountered. The protocol is held until the provider confirms his or her decision to use the system. The provider must specify the level of drug interaction that terminates the protocol. Four levels are available: ≧minimal (level 1), ≧moderate (level 2), ≧major (level 3), and contraindication (level 4). Referring providers must also select their preferred method of communication: e-mail, letters, or both. Finally, they must choose the time between evaluations when their patients are on the maintenance phase.

### IV. Registration

Statin Manager inquires about drug interactions using generic names. Patients will have to ask their pharmacists to label their medications with generic as well as brand names. At entry, they will sign a procedural consent and complete a medication profile (Section VI—Dose Titration Module, Drug Interaction Questions) that will be compared to stop criteria (Section IX—Stop Criteria: Drug Interactions). They become ineligible if statins are contra-indicated with the use of any of their current medications. For other interaction levels, Statin Manager asks for the strength and doses per day and stores this information for future sessions. Patients who pass this step will then undergo a psychological battery that includes screening for depression, alcoholism, and cognitive dysfunction. Baseline laboratory studies include SGOT, SGPT, bilirubin, alkaline phosphatase, CPK, and TSH.

Patients qualifying for self-management will be issued a password to access the site. They will be trained on the use of microcomputers, taught to log-on to the internet, and shown how to access the web site. They will also log-on as 4 hypothetical patients. One lesson each will demonstrate a stop criterion, a hold criterion, an indication for pausing the protocol, and multiple dose adjustments to reach a target LDL. Training will continue until subjects demonstrate their proficiency for all 4 case scenarios. At the conclusion of training, they will register as “live” patients. Registration creates a record in a registration database that includes: patient name; patient number; patient e-mail address; provider e-mail address; age; gender; allowable statins; provider-specified LDL target (if any); past history of angina, MI, stroke, peripheral vascular disease or aortic aneurysm; tobacco use; family history of premature coronary disease; diagnosis of diabetes or hypertension; most recent lipid panel: total cholesterol, total triglycerides, HDL, and LDL, and the entry medication profile. The latter includes drug name, dose, frequency, and total daily dose (dose×frequency). Female gender triggers questions in the algorithm related to possible pregnancy or breast-feeding. Unless the provider specifies a treatment target, the system generates a goal LDL based upon the recommendations of the Adult Treatment Panel III (ATP III). Treatment is initiated, and date rules are then used to generate the dates for the first set of laboratory tests and log-on.

### V. Treatment Targets

Target LDL is based upon the most recommendations of the Adult Treatment Panel III of the National Cholesterol Education Program (21).

### A. Definitions

1. Cardiovascular disease: angina, myocardial infarction, stroke, aortic aneurysm or peripheral vascular disease.
2. Cardiovascular risk factors: age ≧45 years for men and ≧55 years for women; presence of hypertension; tobacco use; most recent HDL <40 mg/dl; occurrence of coronary heart disease in a first-degree (parent, sibling, or child) male relative ≦55 years of age or female relative ≦65 years of age.
3. Number of risk factors: number of cardiovascular risk factors minus 1 (if most recent HDL ≧60 mg/dl)

### B. Target LDL is:

1. <70 mg/dl for patients with diabetes AND cardiovascular disease
2. <100 mg/dl for patients with diabetes OR cardiovascular disease
3. <130 mg/dl for patients with ≧2 cardiovascular risk factors
4. <160 mg/dl for patients with 0 or 1 cardiovascular risk factor

### VI. Dose Titration Module

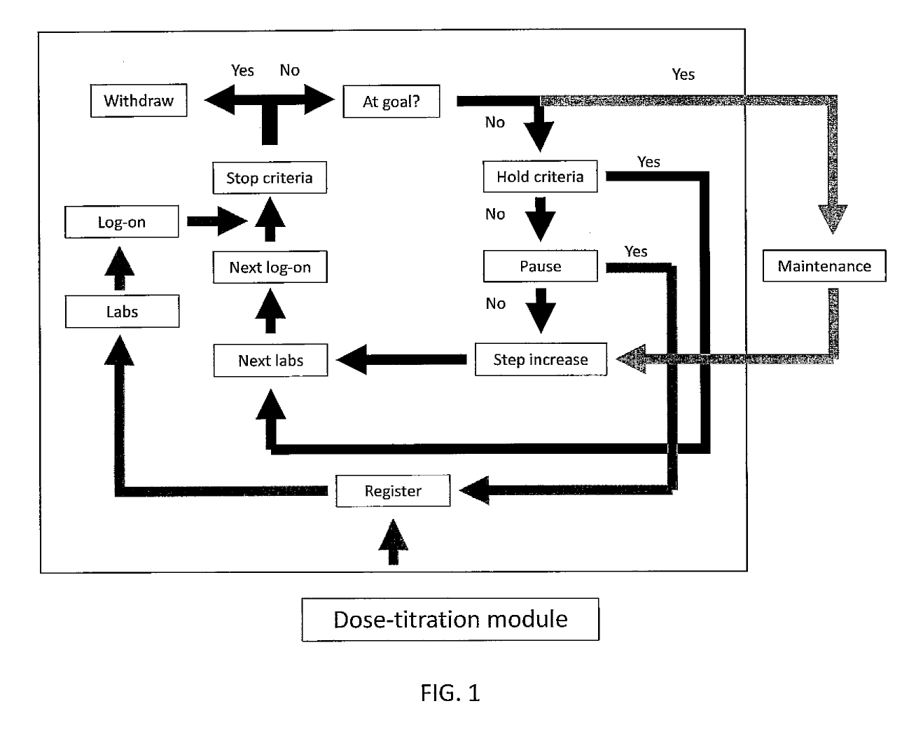

To minimize the risk of toxicity, the Dose Titration Module starts with the lowest recommended dose of the weakest allowable preparation. The patient is guided through several steps until he or she reaches target LDL or the highest recommended dose of the strongest preparation LDL is measured at peak effect for each step, and step increases occur if goal is not reached. The clinical pathway is shown in the FIG. 1.

The Dose Titration Module directs subjects to a series of questions that identify patients who should stop the protocol (Stop Criteria). These questions inquire about disqualifying behaviors or circumstances; previous statin allergies; muscle symptoms; symptoms or signs of liver disease; use of herbals or medications for which statins are contraindicated; pregnancy or breast feeding; co-morbidities that interfere with statin treatment; uncommon, intolerable side effects; and maximal recommended doses for statins in certain combinations. A positive response to any question terminates the protocol. If no such criteria are met, the subject is then presented with his/her most recent laboratory studies. The site determines if there are abnormal liver tests and whether the patient is at goal (At Goal?). Those at goal are instructed to continue with their current medications and are shifted to the Drug Maintenance module. Those not at goal are evaluated to determine if a dose increase should be deferred (Hold Criteria). Deferral is mandated if the subject has been given any drug that increases the risk of statin toxicity or reduces its effectiveness. If no such medications have been prescribed, the subject is asked if he or she will be available for the next step, laboratory test, and log-on. If not, the subject is given the option of withdrawing from the protocol and re-registering when he or she becomes available (Pause Option). If the subject can continue with the protocol, the next medication is prescribed and the next dates for laboratory tests and log-on are set. The site will contain automatic procedures to generate e-mail reminders to patients on the scheduled dates. Treatment and date rules for each step are based upon the relative potency of the preparations and their anticipated maximal effect. The patient continues using the module until a stop criterion is met or the subject meets his or her target LDL. Providers are notified by letter whenever laboratory results become available and step increases occur. When patients log-off, Statin Manager writes all information to the database.

Programming of the Dose Titration Module is based upon the following script. Messages to patients are written in lower case, while subroutines are written in upper case.

### Stop Criteria: Clinical

1. The following questions are about any new symptoms or diagnoses since your last session. Do you want to stop the protocol?
IF “YES”: STOP PROTOCOL
2. Has anyone told you that you are allergic to any of the following statins?
   a. lovastatin or Mevacor
   b. simvastatin or Zocor
   c. pravastatin or Pravachol
   d. fluvastatin or Lescol
   e. atorvastatin or Lipitor
   f. rosuvastatin or Crestor”
IF “YES”: STOP PROTOCOL
3. Do you have muscle pain, aching, soreness, stiffness or weakness?Have you noticed that your urine is reddish or brownish in color?
IF “YES”:
“These symptoms are suggestive of muscle injury. If they are due to your statin, you may have developed a serious complication.”
STOP PROTOCOL
4. Do you have yellowing of your eyes or skin or dark yellow urine?
IF “YES”:
“These symptoms are suggestive of liver injury. If they are due to your statin, you may have developed a serious complication.”
STOP PROTOCOL
5. Have you been diagnosed with liver disease?
IF “YES”: STOP PROTOCOL
6. Has anyone told you that your liver tests are abnormal?
IF “YES”: STOP PROTOCOL
7. Are you now drinking alcohol or using illegal drugs?
IF “YES”: STOP PROTOCOL
8. Have you noticed any hives, skin rash, easy bruising, bleeding, or reddish spots on your skin?
IF “YES”: STOP PROTOCOL
9. For women:
   a. Is there a chance that you could be pregnant?
   b. Are you breast-feeding?
IF “YES”: STOP PROTOCOL
10. The following are uncommon side effects of your statin. Check the ones that have become worse since your last session.
   a. Headache
   b. Diarrhea, constipation or abdominal pain
   c. Passing excess gas
   d. Heartburn
   e. Dizziness, room-spinning or loss of balance
   f. Fatigue or weakness
   g. Nightmares or Inability to sleep
   h. Numbness or tingling of your face, hands or feet
   i. Depression, irritability or memory loss
IF “NO” TO ALL: Go to question 11.
IF “YES” TO ANY: Do they bother you enough to stop the medication?
IF “NO”: Go to question 11.
IF “YES”: STOP PROTOCOL
11. Besides the statin, are you taking any prescription medications, over-the-counter medications, nutritional supplements, herbal medicines, or vitamins?
IF “NO”: Go to 25
IF “YES”: Have you started or increased the dose of any of these medications since your last session? If you're no sure, say “YES”.
IF “NO”: Go to 25
12. Please get all of your nutritional supplements, herbal medicines, and vitamins, if any, and arrange them in alphabetical order. Do you take any nutritional supplements, herbal medicines, or vitamins?
IF “NO”: Go to 12
DISPLAY: You may need to check the labels to answer the following questions. Have you started or increased the dose of any of the following since your last session?
   a. Chaparral
   b. Comfrey
   c. Eucalyptus
   d. Germander
   e. Jin bu huan
   f. Kava
   g. Pennyroyal
   h. Skullcap
   i. Valerian
IF “YES” TO ANY:
“One of your nutritional supplements interferes with your statin treatment.”
STOP PROTOCOL

### Drug Interaction Questions

For Questions 13-25: Display Current Medications with Statin Interactions
13. Gather your prescription and over-the-counter medications, if any, and arrange them in alphabetical order by generic (not brand) name. If you are taking combination pills, you will have to check the bottle for each medication. Are you taking anything for heartburn, acid reflux, gastritis, an ulcer, or upset stomach?
IF “YES”: Are you taking any of the following?
   a. Liquid antacids
   b. Esomeprazole
   c. Magaldrate or simethacone
   d. Omeprazole
14. Are you taking an antibiotic?
IF “YES”: Are you taking any of the following?
   a. Clarithromycin
   b. Dalfopristin
   c. Erythromycin
   d. Fusidic acid
   e. Quinupristin
   f. Rifampin
   g. Telithromycin
   h. Troleandomycin
15. Are you taking anything by mouth to treat a fungal or yeast infection?
IF “YES”: Are you taking any of the following?
   a. Fluconazole
   b. Itraconazole
   c. Ketoconazole
   d. Voriconazole
16. Are you taking any medication for your blood pressure, heart, or lungs?
IF “YES”: Are you taking any of the following?
   a. Amiodarone
   b. Bosentan
   c. Clopidogrel
   d. Diltiazem
   e. Fosphenyloin
   f. Mibefradil
   g. Phenyloin
   h. Verapamil
IF “NO”: Go to 17.
IF “YES” TO 16(a) OR 16(h) AND CURRENT DOSE OF SIMVASTATIN>20 mg:
STOP PROTOCOL
IF “YES” TO 16(a) OR 16(h) AND CURRENT DOSE OF LOVASTATIN>40 mg:
STOP PROTOCOL
IF “YES” TO 16(a) OR 16(h) AND CURRENT DOSE OF SIMVASTATIN=20 mg:
SWITCH TO DRUG MAINTENANCE MODULE
SET DATE FOR NEXT LAB
SET DATE FOR NEXT LOG-ON
DISPLAY: “You have reached the maximum dose of your statin. Your primary care provider will decide whether to increase the dose at this point. You will need to get laboratory tests on [next lab date]. You must fast overnight starting at midnight before getting this test. Your next log-on is scheduled for [next log-on date].”
SET DATE FOR NEXT LAB E-MAIL REMINDER
SET DATE FOR NEXT LOG-ON E-MAIL REMINDER
LETTER TO PRIMARY CARE PROVIDER
PRINT LAB REMINDER FOR [NEXT LAB DATE]
PRINT LOG-ON REMINDER FOR [NEXT LOG-ON DATE]
LOG-OUT
IF “YES” TO 16(a) OR 16(h) AND CURRENT DOSE OF LOVASTATIN=40 mg:
SWITCH TO DRUG MAINTENANCE MODULE
SET DATE FOR NEXT LAB
SET DATE FOR NEXT LOG-ON
DISPLAY: “You have reached the maximum dose of your statin. Your primary care provider will decide whether to increase the dose at this point. You will need to get laboratory tests on [final lab date]. You must fast overnight starting at midnight before getting this test. Your next log-on is scheduled for [next log-on date].”
SET DATE FOR NEXT LAB E-MAIL REMINDER
SET DATE FOR NEXT LOG-ON E-MAIL REMINDER
LETTER TO PRIMARY CARE PROVIDER
PRINT LAB REMINDER FOR [NEXT LAB DATE]
PRINT LOG-ON REMINDER FOR [NEXT LOG-ON DATE]
LOG-OUT
17. Are you taking any medications for diabetes, a hormone condition, or swelling of parts of your body?
IF “YES”: Are you taking any of the following?
   a. Danazol
   b. Exenatide
   c. Troglitazone
18. Do you have HIV or AIDS?
IF “YES”: Are you taking any of the following?
   a. Amprenavir
   b. Atazanavir
   c. Delavirdine
   d. Fosamprenavir
   e. Indinavir
   f. Nelfinavir
   g. Rotinavir
   h. Saquinavir
   i. Tipranavir
19. Besides the statin, are you taking any other medication for your cholesterol or blood lipids?
IF “YES”: Are you taking any of the following?
   a. Bezafibrate
   b. Cholestyramine
   c. Ciprofibrate
   d. Clofibrate
   e. Fenofibrate
   f. Gemfibrozil
   g. Niacin, nicotinic acid, or niaspan?
IF “YES” TO ANY AND SIMVASTATIN DOSE >10 MG:
STOP PROTOCOL
IF “YES” TO ANY AND LOVASTATIN DOSE >20 MG:
STOP PROTOCOL
IF “YES” TO ANY AND SIMVASTATIN DOSE=10 MG:
SWITCH TO DRUG MAINTENANCE MODULE
SET DATE FOR NEXT LAB
SET DATE FOR NEXT LOG-ON
DISPLAY: “You have reached the maximum dose of your statin. Your primary care provider will decide whether to increase the dose at this point. You will need to get laboratory tests on [next lab date]. You must fast overnight starting at midnight before getting this test. Your next log-on is scheduled for [next log-on date].”
SET DATE FOR NEXT LAB E-MAIL REMINDER
SET DATE FOR NEXT LOG-ON E-MAIL REMINDER
LETTER TO PRIMARY CARE PROVIDER
PRINT LAB REMINDER FOR [NEXT LAB DATE]
PRINT LOG-ON REMINDER FOR [NEXT LOG-ON DATE]
LOG-OUT
IF “YES” TO ANY AND LOVASTATIN DOSE=20 MG:
SWITCH TO DRUG MAINTENANCE MODULE
SET DATE FOR NEXT LAB
SET DATE FOR NEXT LOG-ON
DISPLAY: “You have reached the maximum dose of your statin. Your primary care provider will decide whether to increase the dose at this point. You will need to get laboratory tests on [final lab date]. You must fast overnight starting at midnight before getting this test.”
SET DATE FOR NEXT LAB E-MAIL REMINDER
SET DATE FOR NEXT LOG-ON E-MAIL REMINDER
LETTER TO PRIMARY CARE PROVIDER
PRINT LAB REMINDER FOR [NEXT LAB DATE]
PRINT LOG-ON REMINDER FOR [NEXT LOG-ON DATE]
LOG-OUT
20. Do you have an organ transplant (heart, liver, kidneys, or lungs)?
IF “YES”: Are you taking cyclosporine?
21. Do you have leukemia?
IF “YES”: Are you taking imatinib?
22. Are you taking any medication for seizures, nerve damage, or chronic pain?
IF “YES”: Are you taking any of the following?
   a. Fosphenyloin
   b. Carbamazepine
   c. Oxcarbazepine
   d. Phenyloin
23. Are you taking any medication for depression or other mental condition?
IF “YES”: Has anyone started or increased the dose of the following since your last session?
   a. Nefazodone
   b. Risperidone
24. Has anyone started or increased the dose of the following since your last session?
   a. Oat bran
   b. Pectin
   c. St. John's wart
FOR ALL “YES” RESPONSES TO 12-24:
WHAT IS THE DOSE (THE AMOUNT THAT YOU TAKE EACH TIME)?
HOW MANY DOSES DO YOU TAKE PER DAY?
CURRENT DAILY DOSE=DOSE×FREQUENCY
SYSTEM COMPARES CURRENT DAILY DOSE WITH LAST RECORDED DAILY DOSE
WRITE RESPONSE TO MEDICATION RECORD
IF CURRENT DAILY DOSE >LAST DAILY DOSE: RUN DRUG-INTERACTION PROTOCOL
25. We show that your current medication is [current medication]. Is this correct?
IF “NO”: STOP PROTOCOL

### Stop Protocol

“Please stop your statin immediately and call the coordinator”
E-MAIL WARNING TO COORDINATOR
E-MAIL WARNING TO PROVIDER
WITHDRAWN: YES
LOG OUT
26. PROGRAM UPLOADS MOST RECENT LAB RESULTS
We show that you got laboratory tests on [next lab date]. The following are your results.
SGOT ______ (range checking generates warning: SGOT ABNORMAL)
SGPT ______ (range checking generates warning: SGPT ABNORMAL)
CPK ______ (range checking generates warning: CPK ABNORMAL)
LDL
If SGOT, SGPT, OR CPK ABNORMAL:
E-MAIL WARNING TO COORDINATOR
E-MAIL WARNING TO PRIMARY CARE PROVIDER
DISPLAY: “Stop your statin immediately and call the study coordinator”
WITHDRAWN: YES
LOG-OUT

### LDL at Goal

27. IF [(SGOT, SGPT, AND CPK NORMAL) AND LDL AT TARGET:
Switch to Drug Maintenance Module
SET DATE FOR NEXT LAB
SET DATE FOR NEXT SESSION
DISPLAY: “Congratulations! You have met your goal and should remain on this medication. Print the new prescription and take it to the VA pharmacy to get it filled. You should continue on this dose until further notice. Do not forget to get your next laboratory test and log-on one year from now [next lab date]. You must fast overnight starting at midnight before getting this test. Your next log-on is scheduled for [next log-on date].”
SET DATE FOR NEXT LAB E-MAIL REMINDER
SET DATE FOR NEXT SESSION E-MAIL REMINDER
LETTER TO PRIMARY CARE PROVIDER
PRINT LAB REMINDER FOR [NEXT LAB DATE]
PRINT SESSION REMINDER FOR [NEXT SESSION DATE]
PRINT PHARMACY ORDER FOR CURRENT STATIN
PATIENT AT GOAL: YES
LOG-OUT

### LDL not at Goal

28. IF [(SGOT, SGPT, AND CPK NORMAL) and LDL ABOVE TARGET:
DISPLAY: “You have not met your goal. We need to see if your statin dose should be increased at this time. The following questions are about new medications that you may have been given since your last session.”

### Hold Criteria

FOR NON-CRITICAL DRUG INTERACTIONS: HOLD PROTOCOL
29. Do you drink more grapefruit juice since your last session?
Do you drink more than one pint daily?
IF “YES”: HOLD PROTOCOL

### Hold Protocol

SET DATE FOR NEXT LAB
SET DATE FOR NEXT SESSION
DISPLAY: “Some of your medications may alter the effect of your statin. We will not increase the statin dose at this time. You will need to get laboratory tests on [next lab date]. You must fast overnight starting at midnight before getting this test. Your next logon is scheduled for [next session date].”
SET E-MAIL DATE FOR NEXT LAB REMINDER
SET E-MAIL DATE FOR NEXT SESSION REMINDER
LETTER TO PRIMARY CARE PROVIDER
PRINT LAB REMINDER FOR [NEXT LAB DATE]
PRINT SESSION REMINDER FOR [NEXT SESSION DATE]
HOLD: YES
LOG OUT

### Pause Option (Temporary Interruption of Protocol)

30. SET NEXT LAB DATE
SET NEXT SESSION DATE
DISPLAY: “If we change your medication, your next laboratory tests are due for [next lab date] and your next log-on is scheduled for [next session date]. Will you be available on these dates?”
IF “NO”:
DISPLAY: “Continue to take your statin. Call the study coordinator. He or she will review your last blood tests and give you refills for your statin. We will notify your primary care provider that you have temporarily withdrawn from this study. When you become available, call the study coordinator. He or she will register you again for this protocol, order your laboratory tests, and tell you when to log-on again.”
PAUSE: YES
LOG-OUT
LETTER TO PRIMARY CARE PROVIDER

### Step Increase

31. SET NEXT LAB DATE
SET NEXT SESSION DATE
It is OK to increase the dose of your statin at this time. Your next prescription is for:
   Next Medication
Print the prescription and have it filled at the VA pharmacy within the next 2-3 days. When you get the new prescription, throw away the old one. Start taking the new medication the next day. Remember—the most serious side effects are
   a. muscle pain, stiffness, or weakness
   b. yellowing of your eyes and skin or dark urine.
If you notice these symptoms, stop the statin and call the study coordinator right away. You are scheduled for laboratory studies on [next lab date]. You must fast overnight starting at midnight before getting this test. You are scheduled to log-on again on [next session date].
SET DATE FOR NEXT LAB E-MAIL REMINDER
SET DATE FOR NEXT SESSION E-MAIL REMINDER
LETTER TO PRIMARY CARE PROVIDER
PRINT LAB REMINDER FOR [NEXT LAB DATE]
PRINT SESSION REMINDER FOR [NEXT SESSION DATE]
SET CURRENT MEDICATION: NEXT MEDICATION
LOG OUT

### End of Protocol

IF NEXT MEDICATION NOT SPECIFIED:
“You have reached the maximal dose of the strongest statin selected by your provider. Continue with your current medication. Your provider will make additional changes if necessary.”
E-MAIL WARNING TO COORDINATOR
E-MAIL WARNING TO PROVIDER
LOG OUT

### Log Out

WRITE RESPONSES TO DATABASE
LOG OUT

### VII. Drug Maintenance Module

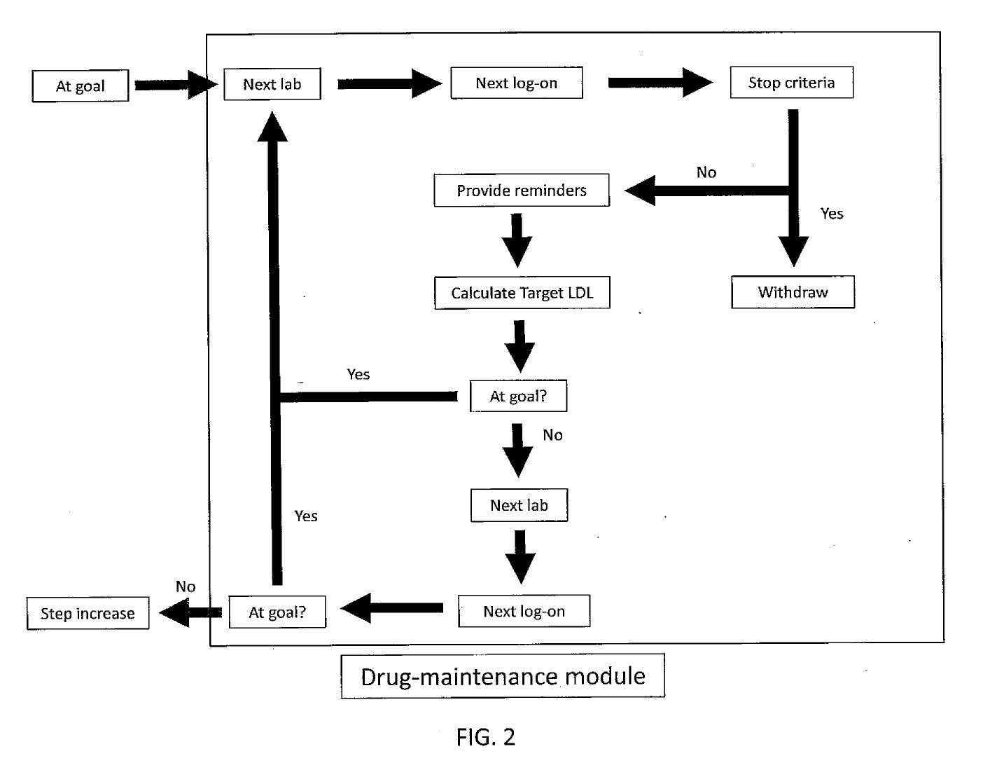

If goal is not reached at the end of the dose-titration protocol, the patient will continue with the last preparation and be referred back to his or her provider. If goal is reached, the patient is switched to the Drug Maintenance Module shown in FIG. 2. Subjects will be scheduled for a periodic evaluation after attaining goal. Again, e-mail reminders will be sent to subjects before these scheduled dates. The patients are again asked about stop criteria identical to those used in the dose-titration module. If they have disqualifying conditions, the patients are told to stop their preparations and referred back to their primary care providers. Those continuing with the protocol are asked if any provider has started a medication with significant statin interactions. If the response to any question is “yes”, an e-mail and/or letter is sent to the primary care providers advising them of the potential drug interactions. Patients are also asked if they have had a macrovascular event or acquired new risk factors in the preceding year. If so, their targets are modified accordingly, and LDL is compared to those standards. If the patient remains at goal, the Drug Maintenance protocol cycles annually. If the subject is not at goal, a message is displayed advising the patient to adhere to his or her medications and laboratory studies are repeated at six weeks. If LDL returns to goal, the patient remains in the drug-maintenance phase. If not, the patient is directed to the Step Increase step of the dose titration module. When the patient logs off the module, all information obtained during the session is written to the database.

Programming of the Drug Maintenance Module is based upon the following script. Again, messages to patients are written in lower case, while subroutines are written in upper case.
1. IF AGE >80: STOP PROTOCOL
2. What is your height?
What is your weight?
CALCULATE BMI
IF BMI<22: STOP PROTOCOL
3. Has anyone ever told you that you have kidney damage?
IF “YES”: STOP PROTOCOL
4. Has anyone told you that you need to take more thyroid medication?
IF “YES”: STOP PROTOCOL
5. Have you had an operation within the last 3 months?
Are you scheduled for an operation in the next 3 months?
IF “YES”: STOP PROTOCOL
6. DEPRESSION SCREEN
IF POSITIVE: STOP PROTOCOL
7. ALCOHOL SCREEN
IF POSITIVE: STOP PROTOCOL
8. MENTAL STATUS TEST
IF POSITIVE: STOP PROTOCOL

### Stop Criteria

RUN STOP QUESTIONS FROM DOSE TITRATION PROTOCOL
FOR “YES”: STOP PROTOCOL

### Provider Reminders

DISPLAY CURRENT MEDICATIONS WITH STATIN INTERACTIONS
RUN DRUG-INTERACTION QUESTIONS FROM DOSE TITRATION PROTOCOL
RUN DRUG-INTERACTION ROUTINE
FOR CRITICAL INTERACTIONS: STOP PROTOCOL

### Stop Protocol

“You are on a medication that may interfere with your statin. You do not have to change your statin at this time. We will notify your primary care provider. Please call the coordinator.”
E-MAIL WARNING TO PRIMARY CARE PROVIDER
E-MAIL WARNING TO COORDINATOR

### Calculate Target LDL

9. Have you had any of the following illnesses in the past year:
   a. a heart attack
   b. angina (chest pain from coronary disease)
   c. stroke
   d. an aneurysm
   e. blockage of blood flow to your legs
IF “YES” TO ANY: RESET CARDIOVASCULAR DISEASE TO YES.
10. Do you have diabetes?
IF “YES”: RESET DIABETES TO YES
11. Do you smoke?
IF “YES”: RESET TOBACCO USE TO YES
12. Do you have high blood pressure?
IF “YES”: RESET HYPERTENSION TO YES
13. Has your father, any brother, or any son had a heart attack before the age of 55?

Has your mother, any sister, or any daughter had a heart attack before the age of 65?
IF “YES” TO 12(a) or 12(b): RESET FAMILY HISTORY TO YES
14. RETRIEVE MOST RECENT HDL
RE-CALCULATE TARGET LDL

### LDL at Goal

15. IF AT GOAL:
SET DATE FOR NEXT LAB (T+[provider-specified interval−7 DAYS])
SET DATE FOR NEXT LOG-ON (T+[provider-specified interval])
Display congratulations message.
Print Reminder for Next Lab
PRINT REMINDER FOR NEXT LOG-ON
SET DATE FOR NEXT LAB E-MAIL REMINDER (T+[provider-specified interval−10 DAYS])
SET DATE FOR NEXT SESSION E-MAIL REMINDER (T+[provider-specified interval−3 DAYS])
Letter to Primary Care Provider
16. IF NOT AT GOAL:
SET DATE FOR NEXT LAB (T+35 DAYS)
SET DATE FOR NEXT SESSION (T+42 DAYS)
“Your LDL is no longer at goal. We will need to repeat your laboratory studies in 6 weeks. We strongly encourage you to take your statin every day during this time. Please get a repeat LDL at [next lab date]. You are scheduled to log-on at [next session date]”.
Print Reminder for Next Lab
PRINT REMINDER FOR NEXT LOG-ON
SET DATE FOR NEXT LAB E-MAIL REMINDER (T+32 DAYS)
SET DATE FOR NEXT SESSION E-MAIL REMINDER (T+39 DAYS)

### Repeat LDL at Goal

17. IF AT GOAL:
SET DATE FOR NEXT LAB (T+[provider-specified interval−7 DAYS])
SET DATE FOR NEXT SESSION (T+[provider-specified interval])
Display congratulations message.
Print Reminder for Next Lab
PRINT REMINDER FOR NEXT SESSION
SET DATE FOR NEXT LAB E-MAIL REMINDER (T+[provider-specified interval−10 DAYS])
SET DATE FOR NEXT SESSION E-MAIL REMINDER (T+[provider-specified interval−3 DAYS)
LETTER TO PRIMARY CARE PROVIDER
18. IF NOT AT GOAL:
Your LDL is still not at goal. We will need to increase your statin treatment at this time
GO TO: STEP INCREASE OF DOSE TITRATION MODULE
LETTER TO PRIMARY CARE PROVIDER

### Log-Out

WRITES RESPONSES TO DATABASE
LOG OUT

### VIII. Drug Interaction Tables

Drug interactions were obtained from MicroMedex DrugDex Evaluations. Medications were reviewed for the severity of the interaction and whether they increased the risk of toxicity or reduced statin effectiveness, seen in Table 1.

TABLE 1
The severity of the interaction of medications and whether they
increased the risk of toxicity or reduced statin effectiveness.

| | Fluva | Lova | Prava | Simva | Atorva | Rosuva |
|---|---|---|---|---|---|---|
| Antacid Group | | | | | | |
| Liquid antacids | | | | | | 2 (−) |
| Esomeprazole | | | | | 3 (+) | |
| Magaldrate | | | | | | 2 (−) |
| Omeprazole | 1 (+) | | | | | |
| Antibiotics | | | | | | |
| Clarithromycin | | 3 (+) | | 3 (+) | 3 (+) | |
| Dalfopristin | 3 (+) | 3 (+) | 3 (+) | 3 (+) | 3 (+) | |
| Erythromycin | 2 (+) | 3 (+) | 2 (+) | 3 (+) | 3 (+) | |
| Fusidic acid | | | | 3 (+) | 3 (+) | |
| Quinupristin | 3 (+) | 3 (+) | 3 (+) | 3 (+) | 3 (+) | |
| Rifampin | 1 (−) | | | 2 (−) | | |
| Telithromycin | | 3 (+) | | 3 (+) | 3 (+) | |
| Troleandomycin | | | | | 3 (+) | |
| Anti-fungals | | | | | | |
| Fluconazole | 1 (+) | 3 (+) | | 3 (+) | 3 (+) | 2 (+) |
| Itraconazole | | 4 (+) | | 4 (+) | 3 (+) | 2 (+) |
| Ketoconazole | | 3 (+) | | 3 (+) | 3 (+) | |
| Voriconazole | | 2 (+) | | 2 (+) | 2 (+) | |
| Cardiovascular Group | | | | | | |
| Amiodarone | | 3 (+) | | 3 (+) | | |
| Bosentan | | 2 (−) | | 2 (−) | 2 (−) | |
| Clopidogrel | 2 (+) | | | | | |
| Diltiazem | | 2 (+) | | 2 (+) | | |
| Fosphenytoin | | | | 2 (−) | 2 (−) | |
| Mibefradil | 1 (+) | 4 (+) | 1 (+) | 4 (+) | 3 (+) | |
| Phenytoin | 2 (+) | | | 2 (−) | 2 (−) | |
| Verapamil | | 3 (+) | | 3 (+) | | |
| Endocrine Group | | | | | | |
| Danazol | | | | 3 (+) | | |
| Exenatide | | 1 (−) | | | | |
| Troglitazone | | | | 1 (−) | | |
| HIV Group | | | | | | |
| Amprenavir | | 3 (+) | 2 (+) | 3 (+) | 2 (+) | |
| Atazanavir | | 3 (+) | | 3 (+) | 3 (+) | |
| Delavirdine | | | | | 2 (+) | |
| Fosamprenavir | | 3 (+) | | 3 (+) | 3 (+) | |
| Indinavir | | 3 (+) | | 3 (+) | 3 (+) | |
| Nelfinavir | | 3 (+) | | 3 (+) | 3 (+) | |
| Rotinavir | | 3 (+) | | 3 (+) | 2 (+) | |
| Saquinavir | | 3 (+) | | 3 (+) | 3 (+) | |
| Tipranavir | | 3 (+) | | 3 (+) | | |
| Hyperlipidemia Group | | | | | | |
| Bezafibrate | 3 (+) | 3 (+) | 3 (+) | 3 (+) | 3 (+) | |
| Cholestyramine | 1 (−) | | 2 (−) | | | |
| Ciprofibrate | 3 (+) | 3 (+) | 3 (+) | 3 (+) | 3 (+) | |
| Clofibrate | 3 (+) | 3 (+) | 3 (+) | 3 (+) | 3 (+) | |
| Colestipol | | | 2 (−) | | | |
| Fenofibrate | 3 (+) | 3 (+) | 3 (+) | 3 (+) | 3 (+) | |
| Gemfibrozil | 3 (+) | 3 (+) | 3 (+) | 3 (+) | 3 (+) | 3 (+) |
| Niacin | 2 (+) | 3 (+) | 2 (+) | 3 (+) | 3 (+) | 3 (+) |
| Immunosuppressives | | | | | | |
| Cyclosporine | 2 (+) | 3 (+) | 2 (+) | 3 (+) | 3 (+) | 3 (+) |
| Imatinib | | | | 1 (+) | | |
| Neurologic Group | | | | | | |
| Fosphenytoin | | | | 2 (−) | 2 (−) | |
| Carbamazepine | | | | 2 (−) | | |
| Oxcarbazepine | | | | 2 (−) | | |
| Phenytoin | 2 (+) | | | 2 (−) | 2 (−) | |
| Psychiatric Group | | | | | | |
| Nefazodone | | 3 (+) | 2 (+) | 3 (+) | 3 (+) | |
| Risperidone | | | | 3 (+) | | |
| Miscellaneous | | | | | | |
| Oat bran | 2 (−) | 2 (−) | 2 (−) | 2 (−) | 2 (−) | 2 (−) |
| Pectin | 2 (−) | 2 (−) | 2 (−) | 2 (−) | 2 (−) | 2 (−) |
| St John's wart | 2 (−) | 2 (−) | 2 (−) | 2 (−) | 2 (−) | 2 (−) |

1 = Minimal
2 = Moderate
3 = Major
4 = Contraindicated
(+) = Toxicity Increased
(−) = Effectiveness Decreased

### IX. Stop Criteria: Drug Interactions

Providers must specify the level of drug interaction that stops the protocol. The options are ≧minimal (1), ≧moderate (2), ≧major (3) or contraindication (4). Table 2 shows the interaction levels for drug combinations that stop the protocol. For example, amprenavir will stop the protocol for pravastatin if the provider is concerned about ≧minimal or ≧moderate interactions but not stop the protocol if he or she is only concerned about ≧major interactions or contraindications.

TABLE 2
Interaction levels for drug combinations that stop the protocol.

| | Fluva | Lova | Prava | Simva | Atorva | Rosuva |
|---|---|---|---|---|---|---|
| Amiodarone | | 1, 2, 3 | | 1, 2, 3 | | |
| Amprenavir | | 1, 2, 3 | 1, 2 | 1, 2, 3 | 1, 2 | |
| Atazanavir | | 1, 2, 3 | | 1, 2, 3 | 1, 2, 3 | |
| Bezafibrate | 1, 2, 3 | 1, 2, 3 | 1, 2, 3 | 1, 2, 3 | 1, 2, 3 | |
| Ciprofibrate | 1, 2, 3 | 1, 2, 3 | 1, 2, 3 | 1, 2, 3 | 1, 2, 3 | |
| Clarithromycin | | 1, 2, 3 | | 1, 2, 3 | 1, 2, 3 | |
| Clofibrate | 1, 2, 3 | 1, 2, 3 | 1, 2, 3 | 1, 2, 3 | 1, 2, 3 | |
| Clopidogrel | 1, 2 | | | | | |
| Cyclosporine | 1, 2 | 1, 2, 3 | 1, 2 | 1, 2, 3 | 1, 2, 3 | 1, 2, 3 |
| Dalfopristin | 1, 2, 3 | 1, 2, 3 | 1, 2, 3 | 1, 2, 3 | 1, 2, 3 | |
| Danazol | | | | 1, 2, 3 | | |
| Delavirdine | | | | | 1, 2 | |
| Diltiazem | | 1, 2 | | 1, 2 | | |
| Erythromycin | 1, 2 | 1, 2, 3 | 1, 2 | 1, 2, 3 | 1, 2, 3 | |
| Esomeprazole | | | | | 1, 2, 3 | |
| Fenofibrate | 1, 2, 3 | 1, 2, 3 | 1, 2, 3 | 1, 2, 3 | 1, 2, 3 | |
| Fluconazole | 1 | 1, 2, 3 | | 1, 2, 3 | 1, 2, 3 | 1, 2 |
| Fosamprenavir | | 1, 2, 3 | | 1, 2, 3 | 1, 2, 3 | |
| Fusidic acid | | | | 1, 2, 3 | 1, 2, 3 | |
| Gemfibrozil | 1, 2, 3 | 1, 2, 3 | 1, 2, 3 | 1, 2, 3 | 1, 2, 3 | 1, 2, 3 |
| Indinavir | | 1, 2, 3 | | 1, 2, 3 | 1, 2, 3 | |
| Itraconazole | | 1, 2, | | 1, 2, | 1, 2, 3 | 1, 2 |
| | | 3, 4 | | 3, 4 | | |
| Ketoconazole | | 1, 2, 3 | | 1, 2, 3 | 1, 2, 3 | |
| Mibefradil | 1 | 1, 2, | 1 | 1, 2, | 1, 2, 3 | |
| | | 3, 4 | | 3, 4 | | |
| Nefazodone | | 1, 2, 3 | 1, 2 | 1, 2, 3 | 1, 2, 3 | |
| Nelfinavir | | 1, 2, 3 | | 1, 2, 3 | 1, 2, 3 | |
| Niacin | 1, 2 | 1, 2, 3 | 1, 2 | 1, 2, 3 | 1, 2, 3 | 1, 2, 3 |
| Omeprazole | 1 | | | | | |
| Phenytoin | 1, 2 | | | | | |
| Quinupristin | 1, 2, 3 | 1, 2, 3 | 1, 2, 3 | 1, 2, 3 | 1, 2, 3 | |
| Risperidone | | | | 1, 2, 3 | | |
| Rotinavir | | 1, 2, 3 | | 1, 2, 3 | 1, 2 | |
| Imatinib | | | | 1 | | |
| Saquinavir | | 1, 2, 3 | | 1, 2, 3 | 1, 2, 3 | |
| Tipranavir | | 1, 2, 3 | | 1, 2, 3 | | |
| Telithromycin | | 1, 2, 3 | | 1, 2, 3 | 1, 2, 3 | |
| Troleando- | | | | | 1, 2, 3 | |
| mycin | | | | | | |
| Verapamil | | 1, 2, 3 | | 1, 2, 3 | | |
| Voriconazole | | 1, 2 | | 1, 2 | 1, 2 | |

### X. Hold Criteria: Drug Interactions

The protocol will be held for drug interactions that do not result in stop criteria whether they increase toxicity or decrease statin effect. Table 3 shows the interaction levels for drug combinations that result in holding the protocol. For example, amprenavir will stop the pravastatin protocol if the provider is concerned ≧minimal or ≧moderate interactions but will result in a hold if he or she is concerned only with ≧major interactions or contraindications.

TABLE 3
Interaction levels for drug combinations that result in
holding the protocol.

| | Fluva | Lova | Prava | Simva | Atorva | Rosuva |
|---|---|---|---|---|---|---|
| Amiodarone | | 4 | | 4 | | |
| Amprenavir | | 4 | 3, 4 | 4 | 3, 4 | |
| Antacids | | | | | | 1, 2, 3, 4 |
| Atazanavir | | 4 | | 4 | 4 | |
| Bezafibrate | 4 | 4 | 4 | 4 | 4 | |
| Bosentan | | 1, 2, | | 1, 2, | 1, 2, | |
| | | 3, 4 | | 3, 4 | 3, 4 | |
| Carbamazepine | | | | 1, 2, | | |
| | | | | 3, 4 | | |
| Cholestyramine | 1, 2, | | 1, 2, | | | |
| | 3, 4 | 3, 4 | | | | |
| Ciprofibrate | 4 | 4 | 4 | 4 | 4 | |
| Clarithromycin | | 4 | | 4 | 4 | |
| Clofibrate | 4 | 4 | 4 | 4 | 4 | |
| Clopidogrel | 3, 4 | | | | | |
| Colestipol | | | 1, 2, | | | |
| | | | 3, 4 | | | |
| Cyclosporine | 3, 4 | 4 | 3, 4 | 4 | 4 | 4 |
| Dalfopristin | 4 | 4 | 4 | 4 | 4 | |
| Danazol | | | | 4 | | |
| Delavirdine | | | | | 3, 4 | |
| Diltiazem | | 3, 4 | | 3, 4 | | |
| Erythromycin | 3, 4 | 4 | 3, 4 | 4 | 4 | |
| Esomeprazole | | | | | 4 | |
| Exenatide | | 1, 2, | | | | |
| | | 3, 4 | | | | |
| Fenofibrate | 4 | 4 | 4 | 4 | 4 | |
| Fluconazole | 2, 3, 4 | 4 | | 4 | 4 | 3, 4 |
| Fosamprenavir | | 4 | | 4 | 4 | |
| Fosphenytoin | | | | 1, 2, | 1, 2, | |
| | | | | 3, 4 | 3, 4 | |
| Fusidic acid | | | | 4 | 4 | |
| Gemfibrozil | 4 | 4 | 4 | 4 | 4 | 4 |
| Indinavir | | 4 | | 4 | 4 | |
| Itraconazole | | | | | 4 | 3, 4 |
| Ketoconazole | | 4 | | 4 | 4 | |
| Mibefradil | 2, 3, 4 | | 2, 3, 4 | | 4 | |
| Nefazodone | | 4 | 3, 4 | 4 | 4 | |
| Nelfinavir | | 4 | | 4 | 4 | |
| Niacin | 3, 4 | 4 | 3, 4 | 4 | 4 | 4 |
| Oat bran | 1, 2, | 1, 2, | 1, 2, | 1, 2, | 1, 2, | 1, 2, |
| | 3, 4 | 3, 4 | 3, 4 | 3, 4 | 3, 4 | 3, 4 |
| Omeprazole | 2, 3, 4 | | | | | |
| Oxcarbazepine | | | | 1, 2, | | |
| | | | | 3, 4 | | |
| Pectin | 1, 2, | 1, 2, | 1, 2, | 1, 2, | 1, 2, | 1, 2, |
| | 3, 4 | 3, 4 | 3, 4 | 3, 4 | 3, 4 | 3, 4 |
| Phenytoin | 3, 4 | | | 1, 2, | 1, 2, | |
| | | | | 3, 4 | 3, 4 | |
| Quinupristin | 4 | 4 | 4 | 4 | 4 | |
| Risperidone | | | | 4 | | |
| Rifampin | 1, 2, | | | 1, 2, | | |
| | 3, 4 | | | 3, 4 | | |
| Rotinavir | | 4 | | 4 | 3, 4 | |
| Imatinib | | | | 2, 3, 4 | | |
| Magaldrate | | | | | | 1, 2, |
| | | | | | | 3, 4 |
| Saquinavir | | 4 | | 4 | 4 | |
| St John's wart | 1, 2, | 1, 2, | 1, 2, | 1, 2, | 1, 2, | 1, 2, |
| | 3, 4 | 3, 4 | 3, 4 | 3, 4 | 3, 4 | 3, 4 |
| Tipranavir | | 4 | | 4 | | |
| Telithromycin | | 4 | | 4 | 4 | |
| Troleando- | | | | | 4 | |
| mycin | | | | | | |
| Verapamil | | 4 | | 4 | | |
| Voriconazole | | 3, 4 | | 3, 4 | 3, 4 | |
| Troglitazone | | | | 1, 2, | | |
| | | | | 3, 4 | | |

### XI. Treatment Rules

Primary care providers may choose the preparations for their patients. The system will automatically generate the dose and type of statin for each step of the Dose Titration module.

A. Single agents—Patients allowed only one statin will start at the initial dose and titrated upward according to the recommendations of MicroMedex. They will be switched to the Drug Maintenance module at the maximal dose even if they have not reached their LDL targets. Letters will be sent to the primary care providers notifying them that they need conversion to another preparation. Tables 4-9 show the steps for each agent:

TABLE 4
The steps for Fluvastatin.
FLUVASTATIN

| Current | Next | Maximal | Switch |
|---|---|---|---|
| Medication | Medication | Dose | Required |
| None | 20 mg | No | No |
| 20 mg | 40 mg | No | No |
| 40 mg | 80 mg | No | No |
| 80 mg | — | Yes | Yes |

TABLE 5
The steps for Simvastatin.
SIMVASTATIN

| Current | Next | Maximal | Switch |
|---|---|---|---|
| Medication | Medication | Dose | Required |
| None | 10 mg | No | No |
| 10 mg | 20 mg | No | No |
| 20 mg | 40 mg | No | No |
| 40 mg | 80 mg | No | No |
| 80 mg | — | Yes | Yes |

TABLE 6
The steps for Lovastatin.
LOVASTATIN

| Current | Next | Maximal | Switch |
|---|---|---|---|
| Medication | Medication | Dose | Required |
| None | 20 mg | No | No |
| 20 mg | 40 mg | No | No |
| 40 mg | 80 mg | No | No |
| 80 mg | — | Yes | Yes |

TABLE 7
The steps for Pravastatin.
PRAVASTATIN

| Current | Next | Maximal | Switch |
|---|---|---|---|
| Medication | Medication | Dose | Required |
| None | 20 mg | No | No |
| 20 mg | 40 mg | No | No |
| 40 mg | 80 mg | No | No |
| 80 mg | — | Yes | Yes |

TABLE 8
The steps for Atorvastatin.
ATORVASTATTN

| Current | Next | Maximal | Switch |
|---|---|---|---|
| Medication | Medication | Dose | Required |
| None | 10 mg | No | No |
| 10 mg | 20 mg | No | No |
| 20 mg | 40 mg | No | No |
| 40 mg | 80 mg | No | No |
| 80 mg | — | Yes | Yes |

TABLE 9
The steps for Rosuvastatin.
ROSUVASTATIN

| Current | Next | Maximal | Switch |
|---|---|---|---|
| Medication | Medication | Dose | Required |
| None | 10 mg | No | No |
| 10 mg | 20 mg | No | No |
| 20 mg | 40 mg | No | No |
| 40 mg | — | Yes | End of protocol |

B. Multiple agents—Information on drug potency is from www.pharmacistsletter.com, an online independent resource supported entirely by its subscribers. The site does not accept support from pharmaceutical companies or advertising. The effect of different doses on LDL is based on evidence derived from over 30 clinical studies and randomized trials. The quality of such evidence is rated from A (for high-quality randomized clinical trials and meta-analyses) to D (for anecdotal experience or animal studies). Table 10 shows the average LDL reduction based on U.S. product labeling and pooled clinical studies (22):

TABLE 10
Average LDL reduction based on U.S. product labeling and
pooled clinical studies.

| Dose | Fluva | Prava | Lova | Simva | Atorva | Rosuva |
|---|---|---|---|---|---|---|
| 5 mg | — | — | — | — | — | 43% |
| 10 mg | — | 19% | — | 28% | 36% | 50% |
| 20 mg | 17% | 24% | 29% | 35% | 46% | 53% |
| 40 mg | 23% | 34% | 31% | 40% | 51% | 62% |
| 80 mg | 33% | 40% | 40-48% | 48% | 54% | — |

To minimize drug toxicity and costs, patients allowed multiple agents will start on the lowest dose of the weakest statin and progress to the highest dose of the strongest statin. The treatment plan depends upon 3 factors: the relative potency of the statins; the recommended dose increments for each statin (above); and a conversion rule when the maximal dose of a given statin is reached. Potency is based upon the mean reduction in LDL for a 20 mg dose in the above table. The following is the rank order (from weakest to strongest): fluvastatin<pravastatin<lovastatin<simvastatin<atorvastatin<rosuvastatin.

Switching to progressively more potent statins will be based upon the following table. The initial dose for the next statin is the one that results in ≧additional 5% reduction in LDL as observed in Table 11.

TABLE 11
Conversion rule for patients on maximal doses of current medication.

| Max dose | Lova | Fluva | Simva | Prava | Atorva | Rosuva |
|---|---|---|---|---|---|---|
| Lovastatin 80 mg | — | none | 80 mg | none | 40 mg | 10 mg |
| Fluvastatin 80 mg | 80 mg | — | 40 mg | 80 mg | 10 mg | 10 mg |
| Simvastatin | none | none | — | none | 80 mg | 20 mg |
| 80 mg | | | | | | |
| Pravastatin 80 mg | 80 mg | none | 80 mg | — | 20 mg | 10 mg |
| Atorvastatin | none | none | none | none | — | 40 mg |
| 80 mg | | | | | | |
| Rosuvastatin | none | none | none | none | None | — |
| 40 mg | | | | | | |

For example, patients failing lovastatin 80 mg daily can only be switched to simvastatin 80 mg, atorvastatin 40 mg, or rosuvastatin 10 mg because they are the only preparations that lower LDL by an additional 5% or more. The new preparation can then be up-titrated until goal is achieved or the maximal dose is reached (when the conversion rule is applied again).

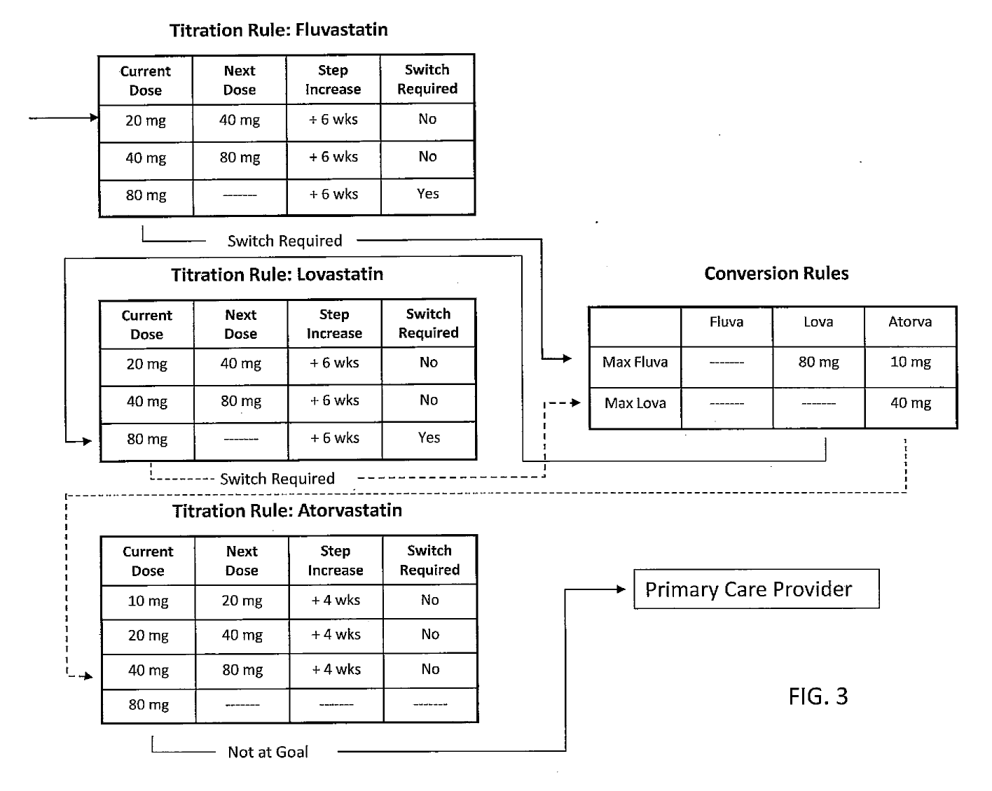

Note that the 3 rules for relative potency, dose titration, and drug conversion define a unique sequence for any combination of allowable statins. In FIG. 3, the referring provider has allowed the use of fluvastatin, lovastatin, and atorvastatin. The protocol starts with fluvastatin (least potent) and ends with atorvastatin (most potent). Dose titrations stop whenever target LDL is reached. Fluvastatin is used at doses of 20 mg, 40 mg, and 80 mg. Because 80 mg is the maximal recommended dose, the conversion rule for fluvastatin is used to identify lovastatin 80 mg as the next dose. If that preparation is not effective, the conversion rule for lovastatin identifies atorvastatin 40 mg as the next step. The patient is then stepped through the 40 and 80 mg doses. If goal has not been reached, the patient is referred back to the primary care provider on the last preparation.

### XII. Date Rules

Date rules govern the timing of letters to providers, e-mail reminders, next laboratory tests, and next sessions for the Dose Titration Module. They are defined by the time to maximal effect for each of the statins and are set after each change in medication (Table 12).

TABLE 12
Date rules.

| Step | Provider | E-mail | Next | E-mail | Next |
|---|---|---|---|---|---|
| Increase | Letter | (Labs) | Lab | (Log-on) | Log-on |
| Lovastatin | time zero | +32 days | +35 days | +39 days | +42 days |
| Fluvastatin | time zero | +18 days | +21 days | +25 days | +28 days |
| Simvastatin | time zero | +32 days | +35 days | +39 days | +42 days |
| Pravastatin | time zero | +18 days | +21 days | +25 days | +28 days |
| Atorvastatin | time zero | +18 days | +21 days | +25 days | +28 days |
| Rosuvastatin | time zero | +18 days | +21 days | +25 days | +28 days |

### XIII. Adherence and Safety Monitoring

A lock-out feature will prevent patients from logging on except for the 7-days following the session date. Site coordinators will monitor patient adherence to the protocol at all times without interrupting self-management. Monitoring will be based upon laboratory and session dates stored in the Dose Titration and Drug Maintenance files. For each workday, the coordinator will extract all laboratory tests due on the preceding 7th day (8th and 9th days for Mondays). If no results are found, a call will be placed to the patient to get the missing laboratory tests. If no tests are found within the next 72 hours, the patient will be dropped from the protocol and primary care provider notified. This precaution assures that no subject will go more than 10-12 days from the first date that a laboratory test is requested. If laboratory results are found, the coordinator will transfer values to a Laboratory file on the web site on that day (beginning of the week for next session). These values will be retrieved and displayed on the appropriate screen when the patient logs on. Finally, the coordinator will review abnormal liver function and muscle enzymes. If any subject with abnormal values fails to call within the 7-9 day window, he or she will be dropped from the protocol and the primary care provider notified. A similar procedure will be used to assure that subjects have logged on at the appropriate times.

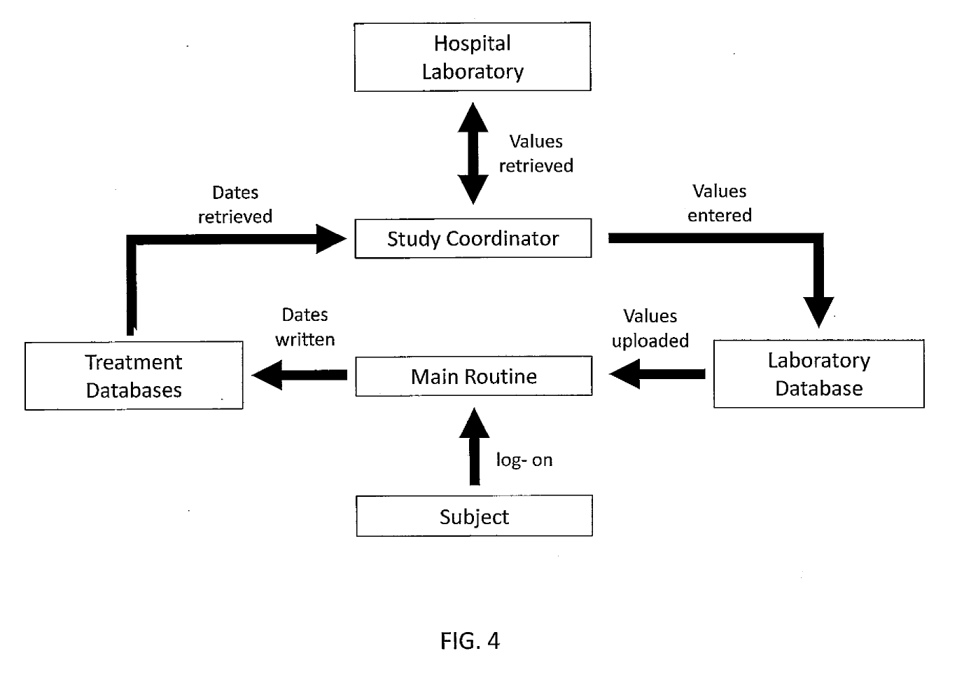

FIG. 4 illustrates this process. The site coordinator will maintain on-line office hours on weekdays. During this time, patients enrolled to the system may log on and enter a chat room with other subjects. Any patient may also request a private session at the end of the public forum. The coordinator will issue bulletins to all subjects if indicated by the chat room discussions.

### XIV. Significance

Statin Manager represents an advance because it is the first internet-based strategy that delivers actual medical treatment (instead of just patient information).

The following summarizes the features of Statin Manager that promote adherence, efficiency, and safety of statin use.

### A. Enhanced Compliance

Requires approval of primary care provider
Option to withdraw at every session
Can call site coordinator at any time
Concurrent website monitoring
E-mail reminders for laboratory tests and sessions
Verbal warning for missed tasks (3-day grace period)
If not at goal on annual review:
advisory to take medications as directed
LDL repeated
then statin dose up-titrated
Patient dropped for non-adherence
Provider notified of missed tasks

### B. Enhanced Efficiency

Log-on at any time and from anywhere
Site coordinator monitors hundreds of patients
Site updated for changes to NCEP guidelines
Site updated for new statins
Site updated for new research findings
Systematic review of risk factors
Target LDL calculated automatically
Option to override LDL target
Sessions and laboratory tests automatically scheduled
E-mail notifications for upcoming tasks
Prints prescriptions and orders for laboratory tests
Treatment plan devised from statins specified by provider
Progresses from lowest dose of weakest statin to highest dose of strongest (minimizes side effects and costs)
Dose up-titrated at peak effect (minimizes time to goal)
Risk factors reviewed, LDL re-calculated, and dose adjusted annually
Provider notified of all actions
No transportation or facility costs
Unburdens provider to discuss lifestyle modification
Future versions interface with hospital computer systems to:
check drug interactions
retrieve laboratory results
write progress notes

### C. Enhanced Safety

Secured website
Lock-out provision to prevent premature use
Algorithms based on DrugDex evaluations (MicroMedex)
LDL targets based upon Adult Treatment Panel III
Stain potency based upon pooled effects in 30 clinical trials
Toxicity monitoring based on joint recommendations of the American College of Cardiology, American Heart Association, and the National Heart, Lung, and Blood Institute
Provider specifies level of drug interactions that terminate protocol
Patients trained on website
Must demonstrate proficiency before using
Screening for rhabdomyolysis and hepatic risk factors at entry
Periodic screening for depression, substance abuse, and cognitive dysfunction
Asks women about pregnancy or breast feeding
Systematically checks for side-effects, drug-interactions, and lab abnormalities
Skips dose titration for new meds with significant interactions
Option to hold protocol for vacations and other delays
Issues warnings on drug interactions to primary care provider
Through iterations, website trains patients on statin management
Chat room and website office hours for problems
Missed tasks and laboratory abnormalities detected within 7-9 days
Minimizes practice variations
Platform for validating conversion rules
The FDA has recently considered statins for over-the-counter status (as in Great Britain). A number of problems were cited in the decision to disapprove this use.
Statin Manager specifically addresses these problems:
   Condition not self-diagnosed
   Prescribed by providers so protocol cannot replace visit
   Continuous involvement by health professional
   Very effective
   Low rate of side-effects
   Treatment effects explicitly evaluated
   Adherence strictly monitored
   Reduces delays in reaching goals
   Thorough review of interactions with other medications
   Improved opportunity for counseling
   No possibility for use by patients who would not benefit
   Prescriptions still covered by insurance
   Database permits population studies
   Statin use monitored annually
   Algorithms reviewed by focus groups for clarity and ease-of-use
   Dose and duration carefully defined
   Contraindications thoroughly reviewed
   No masking of other serious illnesses
   Cannot purchase additional tablets
   Routine questions about pregnancy or breast-feeding
   Possible increase in drug use but cost offset by reduction in macrovascular complications
Still possible for patients to overmedicate or experiment (but no different than routine practice)
Still possible for patients to use as substitute for healthy lifestyle (but frees provider to concentrate on these issues)
If this protocol is successful, the ramifications are substantial. The web-based programs can be adopted by other health care systems and even integrated into their computer systems to facilitate data retrieval and notifications. Alternatively, a commercial site staffed by its own personnel could offer statin titration as a service to busy providers.

### XV. REFERENCES

1. Grant R W, Buse J B, Meigs J B for the University Health System Consortium (UHC) Diabetes Benchmarking Project Team. Quality of diabetes care in U.S. academic medical centers: low rates of medical regimen change. Diabetes Care 2005; 28:337-342.
2. Kennedy A G, MacLean C D, Littenberg B, Ades P A, Pinckney R G. The challenge of achieving national cholesterol goals in patients with diabetes. Diabetes Care 2005; 28:1029-34.
3. Parris E S, Lawrence D B, Mohn L A, Long L B. Adherence to statin therapy and LDL cholesterol goal attainment by patients with diabetes and dyslipidemia. Diabetes Care 2005; 28:595-9.
4. Warsi A, Wang P S, LaValley M P, Avorn J, Solomon D H. Self-management education programs in chronic disease. A systematic review and methodological critique of the literature. Arch Intern Med 2004; 164:1641-9.
5. Newman S, Steed L, Mulligan K. Self-management interventions for chronic illness. Lancet 2004; 364:1523-37.
6. White R H, McCurdy S A, van Marensdorff H, Woodruff D E Jr, Leftgoff L L. Home prothrombin time monitoring after the initiation of warfarin therapy. Ann Intern Med 1989; 11:730-7.
7. Beyth R J, Quinn L, Landefeld C S. A multicomponent intervention to prevent major bleeding complications in older patients receiving warfarin. Ann Intern Med 2000; 133:687-95.
8. de Asis M L, Greene R. A cost-effectivenes analysis of a peak flow-based asthma education and self-management plan in a high-cost population. J Asthma 2004; 41:559-65.
9. Guevara J P, Wolf F M, Grum C M, Clark N M. Effects of educational interventions for self management of asthma in children and adolescents: a systematic review and meta-analysis. BMJ 2003; 326:1308-9.
10. McAlister F A, Stewart S, Ferrua S, McMurray J J J V. Multidisciplinary strategies for the management of heart failure patients at high risk for admission. J Am Coll Cardiol 2004; 44:810-9.
11. Lenhard M J, Reeves G D. Continuous subcutaneous insulin infusion. A comprehensive review of insulin pump therapy. Arch Intern Med 2001; 161:2293-300.
12. Grundy S M, Cleeman J I, Merz N B, Brewer B Jr, Clark L T, Hunninghake D B, Pasternak R C, Smith S C Jr, Stone N J for the Coordinating Committee of the National Cholesterol Education Program. Implications of recent clinical trials for the National Cholesterol Education Program Adult Treatment Panel III guidelines. J Am Coll Cardiol 2004; 44:720-32.
13. Snow V, Aronson M D, Hornbake E R Mottur-Pilson C, Weiss K B for the Clinical Efficacy Assessment Subcommittee for the American College of Physicians. Lipid control in the management of type 2 diabetes mellitus: a clinical practice guideline from the American College of Physicians. Ann Intern Med 2004; 140:644-9.
14. Strom B L. Statins and over-the-counter availability. N Engl J Med 2005; 352:1403-5.
15. Choudhry N K, Avorn J. Over-the-counter statins. Ann Intern Med 2005; 142:910-3.
16. Tate D F, Jackvony E H, Wing R R. Effects of internet behavioral counseling on weight loss in adults at risk for type 2 diabetes. JAMA 2003; 289:1833-6.
17. Lenert L, Munoz R F, Stoddard J. Sansod A, Skoczen S, Perez-Stable E J. Design and pilot evaluation of an internet smoking cessation program. J Am Med Inform Assoc 2003; 10:16-20.
18. McMahon G T, Gomes H E, Hohne S H, Hu T M, Levine B A, Conlin P R. Web-based care management in patients with poorly controlled diabetes. Diabetes Care 2005; 28:1624-9.
19. Kwon H S, Cho J H, Kim H S, Song B R, Ko S H, Lee J M, Kim S R, Chang S A, Kim H S, Cha B Y, Lee K W, Son H Y, Lee J H, Lee W C, Yoon K H. Establishment of blood glucose monitoring system using the internet. Diabetes Care 2004; 27:478-83.
20. Pasternak R C, Smith S C, Bairey-Mierz C N, Grundy S M, Cleeman J I, Lenfant C. ACC/AHA/NHLBI clinical advisory on the use and safety of statins. J Am Coll Cardiol 2002; 40:567-72.
21. Third Report of the National Cholesterol Education Program (NCEP) Expert Panel on detection, evaluation, and treatment of high blood cholesterol in adults (Adult Treatment Panel III). Executive Summary. NIH Publication No. 01-3670. May 2001.
22. Pharmacist's Letter 2003; 19(8):190801.
23. Walsh M N, Simpson R J Jr, Wan G J, Weiss T W, Alexander C M, Markson L E, Berger M L, Pearson T A. Do disease management programs for patients with coronary heart disease make a difference? Experiences of nine practicrs. Am J Managed Care 2002; 8:937-46.
24. Athyros V G, Mikhailidis D P, Papageorgiou A A, Mercouris B R, Athyrou V V, Symeonidis A N, Basayannis E O, Demitriadis D S, Kontopoulos A G. Attaining United Kingdom-European Atherosclerosis Society low-density lipoprotein cholesterol guideline target values in the Greek Atorvastatin and Coronary-heart-disease Evaluation (GREACE) Study. Curr Med Res Opin 2002; 18:499-502.
25. Fanning E L, Selwyn B J, Larme A C, DeFronzo R A. Improving efficacy of diabetes management using treatment algorithms in a mainly Hispanic population. Diabetes Care 2004; 27:1638-46.
26. Ibrahim I A, Beich J, Sidorov J, Gabbay R, Yu L. Measuring outcomes of type 2 diabetes disease management program in an HMO setting. South Med J 2002; 78-87.
27. Gavish D, Leibovitz E, Elly I, Shargorodsky M, Zimlichman R. Follow-up in a lipid clinic improves the management of risk factors in cardiovascular disease patients. Isr Med Assoc J 2002; 4:694-7.
28. Shaffer J, Wexler L F. Reducing low-density lipoprotein cholesterol levels in an ambulatory care system. Results of a multidisciplinary collaborative practice lipid clinic compared with traditional physician-based care. Arch Intern Med 1995; 155:2330-5.
29. Ryan M J Jr, Gibson J, Simmons P, Stanek E. Effectiveness of aggressive management of dyslipidemia in a collaborative-care practice model. Am J Cardiol 2003; 91:1427-31.
30. Yates S, Annis L, Pippins J, Walden S. Does a lipid clinic increase compliance with National Cholesterol Education Program Treatment Guidelines? Report of a case-matched controlled study. South Med J 2001; 94:907-9.
31. Fonarow G C, Gawlinski A, Moughrabi S, Tillisch J H. Improved treatment of coronary heart disease by implementation of a Cardiac Hospital Atherosclerosis Management Program (CHAMP). Am J Cardiol 2001; 87:819-22.
32. Lacy C R, Suh D C, Barone J A, Bueno M, Moylan D, Swartz C, Kudipudi R V, Kostis J B. Impact of a targeted intervention on lipid-lowering therapy in patients with coronary artery disease in the hospital setting. Arch Intern Med 2002; 162:468-73.
33. Khanderia U, Townsend K A, Eagle K, Prager R. Statin initiation following coronary artery bypass grafting: outcome of a hospital discharge protocol. Chest 2005; 127:455-63.
34. Lichtman J H, Amatruda J, Yaari S, Cheng S, Smith G L, Mattera J A, Roumanis S A, Wang Y, Radford M J, Krumholz H M. Clinical trial of an educational intervention to achieve recommended cholesterol levels in patients with coronary artery disease. Am Heart J 2004; 147:522-8.
35. Ghosh S, Aronow W S. Utilization of lipid-lowering drugs in elderly persons with increased serum low-density lipoprotein cholesterol associated with coronary artery disease, symptomatic peripheral arterial disease, prior stroke, or diabetes mellitus before and after an educational program on dyslipidemia treatment. J Gerontol A Biol Sci Med Sci 2003; 58:M432-5.
36. Diwan V K, Wahlstrom R, Tomson G, Beerman B, Sterky G, Eriksson B. Effects of “group detailing” on the prescribing of lipid-lowering drugs: a randomized controlled trial in Swedish primary care. J Clin Epidemiol 1995; 48:705-11.
37. Becker D M, Raqueño J V, Yook R M, Kral B G, Blumenthal R S, Moy T F, Bezirdjian P J, Becker L C. Nurse-mediated cholesterol management compared with enhanced primary care in siblings of individuals with premature coronary disease. Arch Intern Med 1998; 158:1533-9.
38. New J P, Mason J M, Freemantle N, Teasdale S, Wong L M, Bruce N J, Burns J A, Gibson J M. Specialist nurse-led intervention to treat and control hypertension and hyperlipidemia in diabetes (SPLINT). Diabetes Care 2003; 26:2250-5.
39. Rothman R L, Malone R, Bryant B, Shintani A K, Crigler B, Dewalt D A, Dittus R S, Weinberger M, Pignone M P. A randomized trial of a primary care-based disease management program to improve cardiovascular risk factors and glycated hemoglobin levels in patients with diabetes. Am J Med 2005; 118:276-84.
40. Ho P M, Masoudi F A, Peterson E D, Grunwald G K, Sales A E, Hammermeister K E, Rumsfeld J S. Cardiology management improves secondary prevention measures among patients with coronary artery disease. J Am Coll Cardiol 2004; 43:1517-23.
41. Willich N, Müller-Nordhorn J, Sonntag F, Volley H, Meyer-Sabellek W, Wegscheider K, Windier E, Katus H. Economic evaluation of a compliance-enhancing intervention in patients with hypercholesterolemia: design and baseline results of the Open Label Primary Care Study: Rosuvastatin Based Compliance initiatives To Achievements of LDL Goals (ORBITAL) study. Am Heart J 2004; 148:1060-7.
42. Robinson J G, Conroy C, Wickemeyer W J. A novel telephone-based system for management of secondary prevention to a low-density lipoprotein cholesterol ≦100 mg/dl. Am J Cardiol 2000; 85:205-8.
43. Kinn J W, O'Toole M F, Rowley S M, Marek J C, Bufalino V J, Brown A S. Effectiveness of the electronic medical record in cholesterol management in patients with coronary artery disease (Virtual Lipid Clinic). Am J Cardiol 2001; 88:163-5.
44. Grant R W, Cagliero E, Sullivan C M, Dubey A K, Estey G A, Weil E M, Gesmundo J, Nathan D M, Singer D E, Chueh H C, Meigs J B. A controlled trial of population management. Diabetes mellitus: putting evidence into practice (DM-PEP). Diabetes Care 2004; 27:2299-305.

What is claimed is:

1. A method of optimizing therapeutic efficacy for treating hypercholesterolemia in a subject having a cardiovascular disease (CVD), comprising:
(a) determining subject characteristics that affect the likelihood of reaching a goal level of low density lipoprotein (LDL); and
(b) obtaining success probabilities of a variety of statin treatments for reaching said goal level of LDL using said subject characteristics and a multivariate model; and
(c) administrating the optimal statin treatment with the highest success probability of step (b) to said subject thereby optimizing therapeutic efficacy for treating hypercholesterolemia in said subject.

2. A method of optimizing therapeutic efficacy of treatment for lowering the level of LDL in a subject by optimizing therapeutic efficacy for treating hypercholesterolemia using the method of claim 1.

3. A method of predicting the success probability of a statin treatment in a subject having a CVD, comprising:
(a) determining subject characteristics that affect the likelihood of reaching a goal level of LDL; and
(b) inputting said subject characteristics into a multivariate model to obtain the success probability of said statin treatment in said subject.

4. The method of claim 1 or 3, wherein the CVD is atherosclerosis, coronary artery disease, diabetes, cerebrovascular disease, aortic or large vessel disease, or peripheral vascular disease.

5. The method of claim 1 or 3, wherein the statin is any of atorvastatin, ceruvostatin, fluvastatin, lovastatin, osuvastatin, pravastatin, pitavastatin, rosuvastatin, and simvastatin, or a combination thereof.

6. The method of claim 1 or 3, wherein the multivariate model is constructed using a process comprising steps of:
(a) determining subject characteristics of a CAD cohort; and
(b) assembling a predictor file; and
(c) creating a response file; and
(d) linking the predictor file and the response file; and
(e) deriving said multivariate model.

7. The method of claim 1 or 3, wherein the subject characteristics include any one or more of age, gender, BMI, HBA1C (hemoglobin A1c), preceding LDL level, and prior statin dose.

8. The method of claim 7, wherein the subject characteristics further comprises any one of amiodarone dose, carbemazepine dose, pioglitazone dose, diltiazem dose, metformin dose, phenyloin dose, risperidone dose, rosiglitazone dose, and verapamil dose or combination thereof.

9. The method of claim 1 or 3, wherein the goal level of LDL is less than 70 mg/dl.

10. The method of claim 1 or 3, wherein the goal level of LDL is less than 100 mg/dl.
